SEQUENCE LISTING (13 sequences)

SEQ ID NO: 1 - protein, 1271 aa, Artificial Sequence
  Amino acid sequence of full-length S protein
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHY

SEQ ID NO: 2 - protein, 684 aa, Artificial Sequence
  Amino acid sequence of S1 region of S protein
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - protein, 1289 aa, Artificial Sequence
  Amino acid sequence of full-length S gene with IgE leader sequence
MDWTWILFLVAAATRVHSFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHY

SEQ ID NO: 4 - DNA, 3873 bp, Artificial Sequence
  Nucleotide sequence of full-length S gene with IgE leader sequence
atggattggacctggattctgtttctggtggccgctgccacaagagtgcatagcttcgtctttctcgtgctgctgcctctggtgtccagccagtgtgtgaacctgaccacaagaacacagctgcctccagcctacaccaacagctttaccagaggcgtgtactaccccgacaaggtgttcagatctagcgtgctgcacagcacccaggacctgtttctgcccttcttcagcaacgtgacctggttccacgccatccatgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaaaaacaacaagagctggatggaaagcgagttccgggtgtacagcagcgccaacaactgcaccttcgagtacgtgtcccagcctttcctgatggatctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgtaccctgaagtccttcaccgtggaaaagggcatctaccagaccagcaacttcagagtgcagcccaccgagagcatcgtgcggttccccaatatcaccaatctgtgccccttcggcgaggtgttcaatgccaccagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggagtgtctgtgatcacccctggcaccaataccagcaatcaggtggccgtgctgtatcaggacgtgaactgtacagaggtgccagtggccattcacgccgatcagctgacacccacttggagagtgtactccaccggctccaacgtgttccagactagagccggatgtctgatcggagccgagcatgtgaacaacagctacgagtgcgacatccccatcggagctggcatctgtgccagctaccagacacagacaaatagccccagacgggccagaagcgtggcctctcagagcatcattgcctacacaatgagcctgggcgccgagaattctgtggcctacagcaacaactctatcgctatccccaccaacttcaccatcagcgtgaccaccgagatcctgcctgtgtccatgaccaagaccagcgtggactgcaccatgtacatctgcggcgattccaccgagtgcagcaacctgctgctgcagtacggcagcttctgcacccagctgaatagagccctgacagggatcgccgtggaacaggacaagaatacccaagaggtgttcgcccaagtgaagcagatctacaagacccctcctatcaaggacttcggcggcttcaatttcagccagattctgcccgatcctagcaagcccagcaagcggagctttatcgaggacctgctgttcaacaaagtgacactggccgacgccggcttcatcaagcagtatggcgattgcctgggcgacattgccgccagagatctgatttgcgcccagaagtttaacggactgacagtgctgcctcctctgctgaccgatgagatgatcgcccagtacacatctgctctgctggccggcacaatcaccagcggatggacatttggagctggcgcagccctgcagatcccctttgctatgcagatggcctaccggttcaacggcatcggagtgacccagaatgtgctgtacgagaaccagaagctgatcgccaaccagttcaacagcgccatcggcaagatccaggatagcctgtctagcacagccagcgctctgggcaaactgcaggacgtggtcaatcagaacgctcaggccctgaacaccctcgtgaagcagctgagcagcaatttcggcgccatcagctccgtgctgaacgatatcctgagccggctggataaggtggaagccgaggtgcagatcgacagactgatcacaggcagactgcagagcctccagacatacgtgacccagcagctgatcagagccgccgagattagagcctctgccaatctggccgccacaaagatgtctgagtgtgtgctgggccagagcaagagagtggatttctgcggcaagggctaccacctgatgagctttccacagtctgctcctcacggcgtggtgtttctgcatgtgacctacgtgcccgctcaagagaagaacttcacaacagcccctgccatctgccacgacggaaaggcccattttcctagagaaggcgtgttcgtgtccaacggcacccattggttcgtgacacagcggaacttctacgagccccagatcatcaccaccgacaacaccttcgtgtctggcaactgtgacgtcgtgatcggcattgtgaacaataccgtgtacgaccctctgcagcccgagctggacagcttcaaagaggaactggacaagtactttaagaaccacacaagccccgacgtggacctgggcgatattagcggcatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgctgcatgaccagctgctgtagctgcctgaagggctgttgcagctgtggcagctgctgcaagttcgacgaggatgatagcgagcctgtgctgaagggcgtgaaactgcactactaatga

SEQ ID NO: 5 - protein, 1293 aa, Artificial Sequence
  Amino acid sequence of full length S gene with t-PA leader sequence
MDAMKRGLCCVLLLCGAVFVSPFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHY

SEQ ID NO: 6 - DNA, 3885 bp, Artificial Sequence
  Nucleotide sequence of full length S gene with t-PA leader sequence
atggatgctatgaagcgaggactgtgctgcgtgctgctgctgtgtggtgcagtgttcgtgtcccctttcgtgttcctggtcctgctgcctctggtgtccagccagtgtgtgaacctgaccaccagaacacagctgcctccagcctacaccaatagcttcaccaggggcgtgtactaccccgacaaggtgttcagatctagcgtgctgcacagcacccaggacctgtttctgcccttcttcagcaacgtgacctggttccacgccatccatgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggagtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgtaccctgaagtccttcaccgtggaaaagggcatctaccagaccagcaacttcagagtgcagcccaccgagagcatcgtgcggttccccaatatcaccaatctgtgccccttcggcgaggtgttcaatgccacaagatttgccagcgtgtacgcctggaaccggaagagaatcagcaactgcgtggccgactacagcgtgctgtacaatagcgccagcttcagcaccttcaagtgctacggcgtgtcacccaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggcgtgtccgtgatcacacctggcaccaataccagcaatcaggtggccgtgctgtatcaggacgtgaactgtacagaggtgccagtggccattcacgccgatcagctgacacccacttggagagtgtactccaccggctccaacgtgttccagactagagccggatgtctgatcggagccgagcatgtgaacaacagctacgagtgcgacatccccatcggagctggcatctgtgccagctaccagacacagacaaatagccccagacgggccagaagcgtggcctctcagagcatcattgcctacacaatgagcctgggcgccgagaattctgtggcctacagcaacaactctatcgctatccccaccaacttcaccatcagcgtgaccaccgagatcctgcctgtgtccatgaccaagaccagcgtggactgcaccatgtacatctgcggcgattccaccgagtgcagcaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagctttatcgaggacctgctgttcaacaaagtgacactggccgacgccggcttcatcaagcagtatggcgattgcctgggcgacattgccgccagagatctgatttgcgcccagaagtttaacggactgacagtgctgcctcctctgctgaccgatgagatgatcgcccagtacacatctgctctgctggccggcacaatcaccagcggatggacatttggagctggcgcagccctgcagatcccctttgctatgcagatggcctaccggttcaacggcatcggagtgacccagaatgtgctgtacgagaaccagaagctgatcgccaaccagttcaacagcgccatcggcaagatccaggatagcctgtctagcacagccagcgctctgggcaaactgcaggacgtggtcaatcagaacgctcaggccctgaacaccctcgtgaagcagctgagcagcaatttcggcgccatcagctccgtgctgaacgatatcctgagccggctggataaggtggaagccgaggtgcagatcgacagactgatcacaggcagactgcagagcctccagacatacgtgacccagcagctgatcagagccgccgagattagagcctctgccaatctggccgccacaaagatgtctgagtgtgtgctgggccagagcaagagagtggatttctgcggcaagggctaccacctgatgagctttccacagtctgctcctcacggcgtggtgtttctgcatgtgacctacgtgcccgctcaagagaagaacttcacaacagcccctgccatctgccacgacggaaaggcccattttcctagagaaggcgtgttcgtcagcaacggcacccattggttcgtgacacagcggaacttctacgagccccagatcatcaccaccgacaacaccttcgtgtctggcaactgtgacgtcgtgatcggcattgtgaacaataccgtgtacgaccctctgcagcccgagctggacagcttcaaagaggaactggacaagtactttaagaaccacacaagccccgacgtggacctgggcgatattagcggcatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgctgcatgaccagctgctgtagctgcctgaagggctgttgcagctgtggcagctgctgcaagttcgacgaggatgatagcgagcctgtgctgaagggcgtgaaactgcactactaatga

SEQ ID NO: 7 - protein, 702 aa, Artificial Sequence
  Amino acid sequence of S1 region of S gene with IgE leader sequence
MDWTWILFLVAAATRVHSFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 8 - DNA, 2112 bp, Artificial Sequence
  Nucleotide sequence of S1 region of S gene with IgE leader sequence
atggattggacctggattctgtttctggtggccgctgccacaagagtgcatagcttcgtctttctcgtgctgctgcctctggtgtccagccagtgtgtgaacctgaccacaagaacacagctgcctccagcctacaccaacagctttaccagaggcgtgtactaccccgacaaggtgttcagatctagcgtgctgcacagcacccaggacctgtttctgcccttcttcagcaacgtgacctggttccacgccatccatgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaaaaacaacaagagctggatggaaagcgagttccgggtgtacagcagcgccaacaactgcaccttcgagtacgtgtcccagcctttcctgatggatctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgtaccctgaagtccttcaccgtggaaaagggcatctaccagaccagcaacttcagagtgcagcccaccgagagcatcgtgcggttccccaatatcaccaatctgtgccccttcggcgaggtgttcaatgccaccagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggagtgtctgtgatcacccctggcaccaataccagcaatcaggtggccgtgctgtatcaggacgtgaactgtacagaggtgccagtggccattcacgccgatcagctgacacccacttggagagtgtactccaccggctccaacgtgttccagactagagccggatgtctgatcggagccgagcatgtgaacaacagctacgagtgcgacatccccatcggagctggcatctgtgccagctaccagacacagacaaacagccccagacgggccagataatga

SEQ ID NO: 9 - protein, 706 aa, Artificial Sequence
  Amino acid sequence of S1 region of S gene with t-PA leader sequence
MDAMKRGLCCVLLLCGAVFVSPFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 10 - DNA, 2124 bp, Artificial Sequence
  Nucleotide sequence of S1 region of S gene with t-PA leader sequence
atggatgctatgaagcgaggactgtgctgcgtgctgctgctgtgtggtgcagtgttcgtgtcccctttcgtgttcctggtcctgctgcctctggtgtccagccagtgtgtgaacctgaccaccagaacacagctgcctccagcctacaccaatagcttcaccaggggcgtgtactaccccgacaaggtgttcagatctagcgtgctgcacagcacccaggacctgtttctgcccttcttcagcaacgtgacctggttccacgccatccatgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgacccattcctgggagtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgtaccctgaagtccttcaccgtggaaaagggcatctaccagaccagcaacttcagagtgcagcccaccgagagcatcgtgcggttccccaatatcaccaatctgtgccccttcggcgaggtgttcaatgccacaagatttgccagcgtgtacgcctggaaccggaagagaatcagcaactgcgtggccgactacagcgtgctgtacaatagcgccagcttcagcaccttcaagtgctacggcgtgtcacccaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggcgtgtccgtgatcacacctggcaccaataccagcaatcaggtggccgtgctgtatcaggacgtgaactgtacagaggtgccagtggccattcacgccgatcagctgacacccacttggagagtgtactccaccggctccaacgtgttccagactagagccggatgtctgatcggagccgagcatgtgaacaacagctacgagtgcgacatccccatcggagctggcatctgtgccagctaccagacacagacaaacagccccagacgggccagataatga

SEQ ID NO: 11 - protein, 1289 aa, Artificial Sequence
  Amino acid sequence of full-length S gene (Hexapro) with IgE leader sequence
MDWTWILFLVAAATRVHSFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHY

SEQ ID NO: 12 - DNA, 3873 bp, Artificial Sequence
  Nucleotide sequence of full-length S gene (Hexapro) with IgE leader sequence
atggattggacctggattctgtttctggtggccgctgccacaagagtgcacagctttgtctttctcgtgctgctgcctctggtgtccagccagtgtgtgaacctgaccacaagaacacagctgcctccagcctacaccaacagctttaccagaggcgtgtactaccccgacaaggtgttcagatctagcgtgctgcacagcacccaggacctgtttctgcccttcttcagcaacgtgacctggttccacgccatccacgtgtccggcaccaatggcaccaagagattcgacaaccccgtgctgcccttcaacgatggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaaaaacaacaagagctggatggaaagcgagttccgggtgtacagcagcgccaacaactgcaccttcgagtacgtgtcccagcctttcctgatggatctggaaggcaagcagggcaacttcaagaacctgcgcgagttcgtgttcaagaacatcgacggctacttcaagatctacagcaagcacacccctatcaacctcgtgcgggatctgcctcagggcttttctgctctggaacctctggtggacctgcctatcggcatcaacatcacccggtttcagaccctgctggccctgcacagatcttacctgacacctggcgatagcagctctggatggacagctggcgccgctgcctattatgtgggctacctgcagcctcggaccttcctgctgaagtacaacgagaacggcaccatcaccgacgccgtggattgtgctctggatcccctgagcgagacaaagtgcaccctgaagtccttcaccgtggaaaagggcatctaccagaccagcaacttcagagtgcagcccaccgagagcatcgtgcggttccccaatatcaccaatctgtgccccttcggcgaggtgttcaatgccaccagatttgccagcgtgtacgcctggaaccggaagagaatcagcaactgcgtggccgactacagcgtgctgtacaatagcgccagcttcagcaccttcaagtgctacggcgtgtcccctaccaagctgaacgacctgtgcttcaccaatgtgtacgccgacagcttcgtgatcagaggcgacgaagttcggcagatcgctcctggacagacaggcaagatcgccgattacaactacaagctgcccgacgacttcaccggctgcgtgatcgcctggaatagcaacaacctggactccaaagtcggcggcaactacaactacctgtaccggctgttccggaagtccaatctgaagcccttcgagcgggacatctccaccgaaatctatcaggccggcagcaccccttgtaacggcgtggaaggcttcaactgctacttcccactgcagtcctacggctttcagcctaccaatggcgtgggctatcagccctatagagtggtggtgctgagcttcgaactgctgcatgcccctgctaccgtgtgcggccctaagaagtctaccaacctggtcaagaacaaatgcgtgaacttcaacttcaacggcctgaccggcacaggcgtgctgacagagagcaacaagaagttcctgcctttccagcagtttggccgggatatcgccgataccacagacgccgttagagatccccagacactggaaatcctggacatcaccccatgcagctttggcggagtgtctgtgatcacccctggcaccaataccagcaatcaggtggccgtgctgtatcaggacgtgaactgtacagaggtgcccgtggccattcacgccgatcaactgacacccacttggagagtgtactccaccggctccaacgtgttccagactagagccggatgtctgatcggagccgagcacgtgaacaatagctacgagtgcgacatccccatcggcgctggcatctgtgccagctaccagacacagacaaatagccccagacgggccagaagcgtggcctctcagagcatcattgcctacacaatgagcctgggcgccgagaattctgtggcctacagcaacaactctatcgctatccccaccaacttcaccatcagcgtgaccaccgagatcctgcctgtgtccatgaccaagaccagcgtggactgcaccatgtacatctgcggcgattccaccgagtgcagcaacctgctgctgcagtacggcagcttctgcacccagctgaatagagccctgacagggatcgccgtggaacaggacaagaatacccaagaggtgttcgcccaagtgaagcagatctacaagacccctcctatcaaggacttcggcggcttcaatttcagccagattctgcccgatcctagcaagcccagcaagagaagcccaatcgaggacctgctgttcaacaaagtgacactggccgacgccggcttcatcaagcagtatggcgattgcctgggcgacattgccgccagagatctgatttgcgcccagaagtttaacggactgacagtgctgcctcctctgctgaccgatgagatgatcgcccagtacacatctgctctgctggccggcacaatcaccagcggatggacatttggagcaggcccagctctgcagatcccatttccaatgcagatggcctaccggttcaacggcatcggagtgacccagaatgtgctgtacgagaaccagaagctgatcgccaaccagttcaacagcgccatcggcaagatccaggacagcctgtctagcacacctagcgctctgggcaagctgcaggacgtggtcaatcagaacgctcaggccctgaacaccctcgtgaagcagctgagcagcaatttcggcgccatcagctccgtgctgaacgatatcctgagcagactggatcctccagaggccgaggtgcagatcgatagactgatcacaggccggctgcagtccctgcagacatatgtgacacagcagctgatcagagccgccgagattagagcctctgccaatctggccgccacaaagatgtctgagtgtgtgctgggccagagcaagagagtggatttctgcggcaagggctaccacctgatgagctttccacagtctgcccctcacggcgtggtgtttctgcatgtgacatacgtgcccgctcaagagaagaacttcacaacagcccctgccatctgccacgacggaaaggcccattttcctagagaaggcgtgttcgtgtccaacggcacccattggttcgtgacccagcggaacttctacgagccccagatcatcaccaccgacaacaccttcgtgtctggcaactgtgacgtcgtgatcggcattgtgaacaacaccgtgtacgaccctctgcagcccgagctggacagcttcaaagaggaactggacaagtactttaagaaccacacaagccccgacgtggacctgggcgatattagcggcatcaatgcctccgtggtcaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgctgcatgaccagctgttgcagctgcctgaagggctgctgtagctgtggctcctgctgcaagttcgacgaggatgatagcgagcctgtgctgaagggcgtgaaactgcactactaatga

SEQ ID NO: 13 - protein, 1289 aa, Artificial Sequence
  Amino acid sequence of full-length S gene (2P) with IgE leader sequence
MDWTWILFLVAAATRVHSFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHY

(19) United States
(12) Patent Application Publication
(10) Pub. No.: US 2023/0174588 A1
(43) Pub. Date: Jun. 8, 2023

(54) A VACCINE AGAINST SARS-COV-2 AND PREPARATION THEREOF

(71) Applicant: ZYDUS LIFESCIENCES LIMITED, Ahmedabad, Gujarat, (IN)

(72) Inventor: Pankaj PATEL, Ahmedabad, Gujarat (IN)

(21) Appl. No.: 17/920,158

(22) PCT Filed: Apr. 22, 2021
(86) PCT No.: PCT/IB2021/053321

§ 371 Date: Oct. 20, 2022

(30) Foreign Application Priority Data
Apr. 23, 2020 (IN) 202021017410

(51) Int. Cl. C07K 14/005; A61P 31/14

(52) U.S. Cl. CPC C07K 14/005; A61P 31/14; A61K 39/00

(57) ABSTRACT

The current invention provides a DNA construct comprising S gene or S1 gene region of 2019-nCoV spike-S protein. The DNA construct of the present invention comprises DNA plasmid vector carrying S gene or S1 gene region of 2019-nCoV spike-S protein. The vector may further comprise a gene encoding IgE signal peptide or a gene encoding t-PA signal peptide. The DNA construct according to the present invention is further used in the preparation of an immunogenic composition or a vaccine for treating or preventing corona virus or its related diseases.

### FIELD OF THE INVENTION

The present invention relates to a vaccine against SARS-CoV-2. Vaccine according to the present invention is DNA vaccine targeting S gene of novel coronavirus SARS-CoV-2 (2019-nCoV).

### BACKGROUND OF THE INVENTION

Three highly pathogenic human coronaviruses (CoVs) have been identified so far, including Middle East respiratory syndrome coronavirus (MERS-CoV), severe acute respiratory syndrome (SARS) coronavirus (SARS-CoV), and a 2019 novel coronavirus (2019-nCoV), as previously termed by the World Health Organization (WHO). Among them, SARS-CoV was first reported in Guangdong, China in 2002. SARS-CoV caused human-to-human transmission and resulted in the 2003 outbreak with about 10% case fatality rate (CFR), while MERS-CoV was reported in Saudi Arabia in June 2012. Even though with its limited human-to human transmission, MERS-CoV showed a CFR of about 34.4%. The 2019-nCoV was first reported in Wuhan, China in December 2019 from patients with pneumonia, and it has exceeded both SARS-CoV and MERS-CoV in its rate of transmission among humans. 2019-nCoV was renamed SARS-CoV-2 by Coronaviridae Study Group (CSG) of the International Committee on Taxonomy of Viruses (ICTV), while it was renamed HCoV-19, as a common virus name, by a group of virologists in China. The disease and the virus causing it were named Coronavirus Disease 2019 (COVID-19) and the virus responsible for COVID-19 or the COVID-19 virus, respectively, by WHO. The outbreak of a novel coronavirus (2019-nCoV) represents a pandemic threat that has been declared a public health emergency of international concern (PHEIC) (1). As of Apr. 21, 2020, a total of 24, 99,665 confirmed cases of COVID-19 were reported, including 1, 71,338 deaths globally, in China, Europe, USA, India and at least 200 other countries and/or territories (2). Currently, the intermediate host of SARS-CoV-2 is still unknown, and no effective prophylactics or therapeutics are available. It shows an urgent need for the immediate development of vaccines and antiviral drugs for prevention and treatment of COVID-19 (1). More than 100 pre-clinical or clinical trials are going on, which include repurposing of already approved drugs but with different indications such as anti-malarial, anti-viral, anti-parasitic drugs, cytokine or complement targeting antibodies, etc. However, these drugs may help to prevent worsening of the coronavirus infection. Still, there is an unmet need of a vaccine against novel coronavirus SARS-CoV-2. Current invention provides a DNA construct and its composition which can be developed as a vaccine against SARS-CoV-2. A coronavirus contains four structural proteins, including spike (S), envelope (E), membrane (M), and nucleocapsid (N) proteins. Among them, S protein plays the most important roles in viral attachment, fusion and entry, and it serves as a target for development of antibodies, entry inhibitors and vaccines. The S protein mediates viral entry into host cells by first binding to a host receptor through the receptor-binding domain (RBD) in the S1 subunit and then fusing the viral and host membranes through the S2 subunit. SARS-CoV recognizes angiotensin-converting enzyme 2 (ACE2) as its receptor. Similar to SARS-CoV, SARS-CoV-2 also recognizes ACE2 as its host receptor binding to viral S protein (1). A 3.5-angstrom-resolution cryo-electron microscopy structure of the 2019-nCoV S trimer in the prefusion conformation is recently published in reference 3 which is incorporated herein the present application. According to this recent study, the predominant state of the trimer has one of the three receptor-binding domains (RBDs) rotated up in a receptor-accessible conformation. Biophysical and structural evidence suggests that the 2019-nCoV S protein binds angiotensin-converting enzyme 2 (ACE2) with higher affinity than does severe acute respiratory syndrome (SARS)-CoV S. Additionally, several published SARS-CoV RBD-specific monoclonal antibodies were tested and found that they do not have appreciable binding to 2019-nCoV S, suggesting that antibody cross-reactivity may be limited between the two RBDs. It shows that S protein of the SARS-CoV-2 is very unique and cannot be inhibited by the conventional antibodies or other therapeutics which can inhibit S protein of conventional coronavirus (3). However, bamlanivimab and the combination of casirivimab plus imdevimab are developed that are specific anti-severe acute respiratory syndrome coronavirus 2 (SARS-CoV-2) monoclonal antibodies available through Food and Drug Administration (FDA) Emergency Use Authorizations (EUAs) for the treatment of outpatients with mild to moderate COVID-19 who are high risk for progressing to severe disease and/or hospitalization.

Here in the present application, current invention provides novel construct comprising DNA plasmid vector carrying S gene which encodes spike-S protein of 2019-nCoV or S1 gene region of said S gene. The novel DNA construct of the present invention can be developed as a vaccine for preventing or treating coronavirus or its related disease.

Three candidates including two mRNA based candidate from Pfizer and Moderna and Chimpanzee adenovirus vector based candidate from AstraZeneca got emergency use authorization. The emergency use was approved based on Phase-3 efficacy data. The mRNA vaccines from Pfizer reported 95% efficacy (10), whereas Moderna and AstraZeneca reported 94.5% and 70.4% efficacy respectively for their vaccine candidate. Further, Sputnik V vaccine with 92% efficacy developed at the National Research Centre for Epidemiology and Microbiology (11).

The conventional active vaccines are made of a killed or attenuated form of the infectious agent. Vaccination with live attenuated and killed vaccines in most cases results in generation of humoral but not a cell-mediated immune response. What is required in such cases, but not available, are antigens that are safe to use, that can be processed by the endogenous pathway and eventually activating both B and T cell response. The activated lymphocytes generated would destroy the pathogen-infected cell. For these reasons, a new approach of vaccination that involves the injection of a piece of DNA that contains the genes for the antigens of interest are under investigation. DNA vaccines are attractive because they ensure appropriate folding of the polypeptide, produce the antigen over long periods, and do not require adjuvants. These host-synthesized antigens then can become the subject of immune surveillance in the context of both major histocompatibility complex class I (MHC I) and MHC II proteins of the vaccinated individual (12). By contrast, standard vaccine antigens are taken up into cells by phagocytosis or endocytosis and are processed through the MHC class II system, which primarily stimulates antibody response. In addition to these properties, the plasmid vector contains immunostimulatory nucleotide sequences—unmethylated cytidine phosphate guanosine (CpG) motifs—that induce strong cellular immunity (13). Finally, DNA vaccine has shown to stimulate sustained immune responses.

### SUMMARY OF THE INVENTION

The current invention provides a DNA construct comprising S gene which encodes spike-S protein of 2019-nCoV or S1 gene region of said S gene. The DNA construct of the present invention comprises DNA plasmid vector carrying S gene which encodes spike-S protein of 2019-nCoV or S1 gene region of said S gene. The preferred vector according to the current invention is pVAX1. In another aspect, the vector may further comprise a gene encoding IgE signal peptide or a gene encoding t-PA signal peptide. The DNA construct according to the present invention is further used in the preparation of an immunogenic composition or a vaccine for treating or preventing corona virus or its related diseases. The SARS-CoV-2 DNA vaccine will need to be supplied in multi-million doses. In order to achieve it, scalable, efficient and cost effective process with shortened process time and a process that can provide high recovery of plasmid DNA is illustrated herein the present application. In certain aspect, the DNA construct prepared according to the current invention is administered into muscles cells or injected intradermally into the subject. In one of such aspects, the administration of DNA construct into muscles cell is performed by needle-free injector system or by electroporator system. Further, in order to improve uptake efficacy of DNA vaccine into muscle cells, different formulations comprising one or more of water, saline, buffers, stabilizers, adjuvants, excipients and lipid formulations can be used to prepare an immunogenic composition of the present invention for treating or preventing COVID-19 or its related diseases. In another one of such aspects, the intra-dermal administration of DNA construct is performed by needle-free injector system and/or by electroporator system. Further, in order to improve uptake efficacy of DNA vaccine into muscle cells, different formulation with buffers, stabilizers, adjuvants, excipients and lipid formulations can be used to prepare an immunogenic composition of the present invention for treating or preventing COVID-19 or its related diseases.

### BRIEF DESCRIPTION OF FIGURES

FIG. 1: It depicts vector map of pVAX1 vector carrying full length S gene with IgE leader sequence

FIG. 2: It depicts vector map of pVAX1 vector carrying full length S gene with t-PA leader sequence

FIG. 3: It depicts vector map of pVAX1 vector carrying S1 region of S gene with IgE leader sequence

FIG. 4: It depicts vector map of pVAX1 vector carrying S1 region of S gene with t-PA leader sequence

FIG. 5a: It depicts fluorescence images showing expression of S protein after transfection of Vero cells with DNA construct comprising S gene of SARS-CoV-2 or empty plasmid (control) by immunofluorescence

FIG. 5b: It depicts fluorescence images showing expression of S1 protein after transfection of Vero cells with DNA construct comprising S1 gene of SARS-CoV-2 or empty plasmid (control) by immunofluorescence

FIG. 6: It depicts antibody response after DNA vaccination in BALB/c mice and long term immunogenicity

FIG. 7: It depicts antibody response after DNA vaccination in Guinea Pigs

FIG. 8: It depicts IFN-γ responses in BALB/c mice post-administration of DNA vaccine

List of nucleotide sequences and amino acid sequences of the present invention

SEQ ID No.: 1-Amino acid sequence of full-length S protein
FVFLVLLPLVSSQCVNLTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSLLIVNNATNVVIKVC
EFQFCNDPFLGVYYHKNNKSWMESEFRVYSSANNCTFEYVSQPFLMDLEGKQGNFKNLREFVFKN
IDGYFKIYSKHTPINLVRDLPQGFSALEPLVDLPIGINITRFQTLLALHRSYLTPGDSSSGWTAG
AAAYYVGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQPYRVVVLSFELLHAP
ATVCGPKKSTNLVKNKCVNFNFNGLTGTGVLTESNKKFLPFQQFGRDIADTTDAVRDPQTLEILD
ITPCSFGGVSVITPGTNTSNQVAVLYQDVNCTEVPVAIHADQLTPTWRVYSTGSNVFQTRAGCLI
GAEHVNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDKNTQEV
EAQVKQLYKTPELKDFGGMNESQLLPDPSKPSKRSELEDLLHNKVTLADAGELKQYGDCLGDLAA
RDLICAQKFNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSSNFGAISSV
LNDILSRLDKVEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDSFKEELDKYFKNHTSPDVDLGDISG
INASVVNIQKEIDRLNEVAKNLNESLIDLQELGKYEQYIKWPWYIWLGFIAGLIAIVMVTIMLCC
MTSCCSCLKGCCSCGSCCKFDEDDSEPVIKGVKLHY
SEQ ID No.: 2-Amino acid sequence of S1 region of S protein
EVFLVLLPLVSSQCVNLTTRTQLPPAYTNSETRGVYYPDKVFRSSVLHSTQDLFLPFFSNVTWFH
AIHVSGTNGTKRFDNPVLPFNDGVYFASTEKSNIIRGWIFGTTLDSKTQSLLIVNNATNVVIKVC
EFQFCNDPFLGVYYHKNNKSWMESEFRVYSSANNCTFEYVSQPFLMDLEGKQGNFKNLREFVFKN
IDGYFKIYSKHTPINLVRDLPQGFSALEPLVDLPIGINITRFQTLLALHRSYLTPGDSSSGWTAG
AAAYYVGYLQPRTFLLKYNENGTITDAVDCALDPLSETKCTLKSFTVEKGIYQTSNFRVQPTESI
VRFPNITNLCPFGEVFNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQPYRVVVLSFELLHAP
ATVCGPKKSTNLVKNKCVNFNFNGLTGTGVLTESNKKFLPFQQFGRDIADTTDAVRDPQTLEILD
ITPCSFGGVSVITPGTNTSNQVAVLYQDVNCTEVPVAIHADQLTPTWRVYSTGSNVFQTRAGCLI
GAEHVNNSYECDIPIGAGICASYQTOTNSPRRAR
SEQ ID NO.: 3-Amino acid sequence of full-length S gene with IgE leader sequence
MDWTWILFLVAAATRVHSFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTV
EKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSIIAYTMS
LGAENSVAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSFIEDLLFNKVTL
ADAGFIKQYGDCLGDIAARDLICAQKFNGLTVLPPLLTDEMIAQYTSALLAGTITSGWTFGAGAA
LQIPFAMQMAYRFNGIGVTQNVLYENQKLIANQFNSAIGKIQDSLSSTASALGKLQDVVNQNAQA
LNTLVKQLSSNFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKWPWYIWL
GFIAGLIAIVMVTIMLCCMTSCCSCLKGCCSCGSCCKFDEDDSEPVLKGVKLHY
1-18 underlined amino acid residues represent amino acid sequence of IgE leader
sequence and 19-1289 amino acid residues represent amino acid sequence of full-
length S protein.
SEQ ID NO.: 4-Nucleotide sequence of full-length S gene with IgE leader sequence
ATGGATTGGACCTGGATTCTGTTTCTGGTGGCCGCTGCCACAAGAGTGCATAGCTTCGTCTTTCT
CGTGCTGCTGCCTCTGGTGTCCAGCCAGTGTGTGAACCTGACCACAAGAACACAGCTGCCTCCAG
CCTACACCAACAGCTTTACCAGAGGCGTGTACTACCCCGACAAGGTGTTCAGATCTAGCGTGCTG
CACAGCACCCAGGACCTGTTTCTGCCCTTCTTCAGCAACGTGACCTGGTTCCACGCCATCCATGT
GTCCGGCACCAATGGCACCAAGAGATTCGACAACCCCGTGCTGCCCTTCAACGATGGGGTGTACT
TTGCCAGCACCGAGAAGTCCAACATCATCAGAGGCTGGATCTTCGGCACCACACTGGACAGCAAG
ACCCAGAGCCTGCTGATCGTGAACAACGCCACCAACGTGGTCATCAAAGTGTGCGAGTTCCAGTT
CTGCAACGACCCCTTCCTGGGCGTCTACTACCACAAAAACAACAAGAGCTGGATGGAAAGCGAGT
TCCGGGTGTACAGCAGCGCCAACAACTGCACCTTCGAGTACGTGTCCCAGCCTTTCCTGATGGAT
CTGGAAGGCAAGCAGGGCAACTTCAAGAACCTGCGCGAGTTCGTGTTCAAGAACATCGACGGCTA
CTTCAAGATCTACAGCAAGCACACCCCTATCAACCTCGTGCGGGATCTGCCTCAGGGCTTTTCTG
CTCTGGAACCTCTGGTGGACCTGCCTATCGGCATCAACATCACCCGGTTTCAGACCCTGCTGGCC
CTGCACAGATCTTACCTGACACCTGGCGATAGCAGCTCTGGATGGACAGCTGGCGCCGCTGCCTA
TTATGTGGGCTACCTGCAGCCTCGGACCTTCCTGCTGAAGTACAACGAGAACGGCACCATCACCG
ACGCCGTGGATTGTGCTCTGGATCCCCTGAGCGAGACAAAGTGTACCCTGAAGTCCTTCACCGTG
GAAAAGGGCATCTACCAGACCAGCAACTTCAGAGTGCAGCCCACCGAGAGCATCGTGCGGTTCCC
CAATATCACCAATCTGTGCCCCTTCGGCGAGGTGTTCAATGCCACCAGATTTGCCAGCGTGTACG
CCTGGAACCGGAAGAGAATCAGCAACTGCGTGGCCGACTACAGCGTGCTGTACAATAGCGCCAGC
TTCAGCACCTTCAAGTGCTACGGCGTGTCCCCTACCAAGCTGAACGACCTGTGCTTCACCAATGT
GTACGCCGACAGCTTCGTGATCAGAGGCGACGAAGTTCGGCAGATCGCTCCTGGACAGACAGGCA
AGATCGCCGATTACAACTACAAGCTGCCCGACGACTTCACCGGCTGCGTGATCGCCTGGAATAGC
AACAACCTGGACTCCAAAGTCGGCGGCAACTACAACTACCTGTACCGGCTGTTCCGGAAGTCCAA
TCTGAAGCCCTTCGAGCGGGACATCTCCACCGAAATCTATCAGGCCGGCAGCACCCCTTGTAACG
GCGTGGAAGGCTTCAACTGCTACTTCCCACTGCAGTCCTACGGCTTTCAGCCTACCAATGGCGTG
GGCTATCAGCCCTATAGAGTGGTGGTGCTGAGCTTCGAACTGCTGCATGCCCCTGCTACCGTGTG
CGGCCCTAAGAAGTCTACCAACCTGGTCAAGAACAAATGCGTGAACTTCAACTTCAACGGCCTGA
CCGGCACAGGCGTGCTGACAGAGAGCAACAAGAAGTTCCTGCCTTTCCAGCAGTTTGGCCGGGAT
ATCGCCGATACCACAGACGCCGTTAGAGATCCCCAGACACTGGAAATCCTGGACATCACCCCATG
CAGCTTTGGCGGAGTGTCTGTGATCACCCCTGGCACCAATACCAGCAATCAGGTGGCCGTGCTGT
ATCAGGACGTGAACTGTACAGAGGTGCCAGTGGCCATTCACGCCGATCAGCTGACACCCACTTGG
AGAGTGTACTCCACCGGCTCCAACGTGTTCCAGACTAGAGCCGGATGTCTGATCGGAGCCGAGCA
TGTGAACAACAGCTACGAGTGCGACATCCCCATCGGAGCTGGCATCTGTGCCAGCTACCAGACAC
AGACAAATAGCCCCAGACGGGCCAGAAGCGTGGCCTCTCAGAGCATCATTGCCTACACAATGAGC
CTGGGCGCCGAGAATTCTGTGGCCTACAGCAACAACTCTATCGCTATCCCCACCAACTTCACCAT
CAGCGTGACCACCGAGATCCTGCCTGTGTCCATGACCAAGACCAGCGTGGACTGCACCATGTACA
TCTGCGGCGATTCCACCGAGTGCAGCAACCTGCTGCTGCAGTACGGCAGCTTCTGCACCCAGCTG
AATAGAGCCCTGACAGGGATCGCCGTGGAACAGGACAAGAATACCCAAGAGGTGTTCGCCCAAGT
GAAGCAGATCTACAAGACCCCTCCTATCAAGGACTTCGGCGGCTTCAATTTCAGCCAGATTCTGC
CCGATCCTAGCAAGCCCAGCAAGCGGAGCTTTATCGAGGACCTGCTGTTCAACAAAGTGACACTG
GCCGACGCCGGCTTCATCAAGCAGTATGGCGATTGCCTGGGCGACATTGCCGCCAGAGATCTGAT
TTGCGCCCAGAAGTTTAACGGACTGACAGTGCTGCCTCCTCTGCTGACCGATGAGATGATCGCCC
AGTACACATCTGCTCTGCTGGCCGGCACAATCACCAGCGGATGGACATTTGGAGCTGGCGCAGCC
CTGCAGATCCCCTTTGCTATGCAGATGGCCTACCGGTTCAACGGCATCGGAGTGACCCAGAATGT
GCTGTACGAGAACCAGAAGCTGATCGCCAACCAGTTCAACAGCGCCATCGGCAAGATCCAGGATA
GCCTGTCTAGCACAGCCAGCGCTCTGGGCAAACTGCAGGACGTGGTCAATCAGAACGCTCAGGCC
CTGAACACCCTCGTGAAGCAGCTGAGCAGCAATTTCGGCGCCATCAGCTCCGTGCTGAACGATAT
CCTGAGCCGGCTGGATAAGGTGGAAGCCGAGGTGCAGATCGACAGACTGATCACAGGCAGACTGC
AGAGCCTCCAGACATACGTGACCCAGCAGCTGATCAGAGCCGCCGAGATTAGAGCCTCTGCCAAT
CTGGCCGCCACAAAGATGTCTGAGTGTGTGCTGGGCCAGAGCAAGAGAGTGGATTTCTGCGGCAA
GGGCTACCACCTGATGAGCTTTCCACAGTCTGCTCCTCACGGCGTGGTGTTTCTGCATGTGACCT
ACGTGCCCGCTCAAGAGAAGAACTTCACAACAGCCCCTGCCATCTGCCACGACGGAAAGGCCCAT
TTTCCTAGAGAAGGCGTGTTCGTGTCCAACGGCACCCATTGGTTCGTGACACAGCGGAACTTCTA
CGAGCCCCAGATCATCACCACCGACAACACCTTCGTGTCTGGCAACTGTGACGTCGTGATCGGCA
TTGTGAACAATACCGTGTACGACCCTCTGCAGCCCGAGCTGGACAGCTTCAAAGAGGAACTGGAC
AAGTACTTTAAGAACCACACAAGCCCCGACGTGGACCTGGGCGATATTAGCGGCATCAATGCCAG
CGTCGTGAACATCCAGAAAGAGATCGACCGGCTGAACGAGGTGGCCAAGAATCTGAACGAGAGCC
TGATCGACCTGCAAGAACTGGGGAAGTACGAGCAGTACATCAAGTGGCCCTGGTACATCTGGCTG
GGCTTTATCGCCGGACTGATTGCCATCGTGATGGTCACAATCATGCTGTGCTGCATGACCAGCTG
CTGTAGCTGCCTGAAGGGCTGTTGCAGCTGTGGCAGCTGCTGCAAGTTCGACGAGGATGATAGCG
AGCCTGTGCTGAAGGGCGTGAAACTGCACTACTAATGA
1-54 underlined nucleotide residues represent DNA sequence (nucleotide sequence)
of IgE leader sequence and 55-3873 nucleotide residues represent DNA sequence
(nucleotide sequence) of S gene.
SEQ ID NO.: 5-Amino acid sequence of full length S gene with t-PA leader sequence
MDAMKRGLCCVLLLCGAVFVSPFVFLVLLPLVSSQCVNLTTRTQLPPAYTNSFTRGVYYPDKVFR
SSVLHSTQDLFLPFFSNVTWFHAIHVSGTNGTKRFDNPVLPFNDGVYFASTEKSNIIRGWIFGTT
LDSKTQSLLIVNNATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKCTLK
SFTVEKGIYQTSNFRVQPTESIVRFPNITNLCPFGEVFNATRFASVYAWNRKRISNCVADYSVIY
NSASFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYGFQP
TNGVGYQPYRVVVLSFELLHAPATVCGPKKSTNLVKNKCVNFNFNGLTGTGVLTESNKKFLPFQQ
FGRDIADTTDAVRDPQTLEILDITPCSFGGVSVITPGTNTSNQVAVLYQDVNCTEVPVAIHADQL
TPTWRVYSTGSNVFQTRAGCLIGAEHVNNSYECDIPIGAGICASYQTQTNSPRRARSVASQSIIA
YTMSLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDLLFN
KVTLADAGFIKQYGDCLGDIAARDLICAQKFNGLTVLPPLLTDEMIAQYTSALLAGTITSGWTFG
AGAALQIPFAMQMAYRFNGIGVTQNVLYENQKLIANQFNSAIGKIQDSLSSTASALGKLQDVVNQ
NAQALNTLVKQLSSNFGAISSVLNDILSRLDKVEAEVAIDRLITGRLQSLQTYVTQQLIRAAEIR
ASANLAATKMSECVLGQSKRVDFCGKGYHLMSFPQSAPHGVVFLHVTYVPAQEKNFTTAPAICHD
GKAHFPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQYIKWPW
YIWLGFTAGLIAIVMVTIMLCCMTSCCSCLKGCCSCGSCCKFDEDDSEPVLKGVKLHY
1-22 underlined amino acid residues represent ammo acid sequence of t-PA leader
sequence and 23-1293 amino acid residues represent amino acid sequence of full-
length S protein.
SEQ ID NO.: 6-Nucleotide sequence of full length S gene with t-PA leader sequence
ATGGATGCTATGAAGCGAGGACTGTGCTGCGTGCTGCTGCTGTGTGGTGCAGTGTTCGTGTCCCC
TTTCGTGTTCCTGGTCCTGCTGCCTCTGGTGTCCAGCCAGTGTGTGAACCTGACCACCAGAACAC
AGCTGCCTCCAGCCTACACCAATAGCTTCACCAGGGGCGTGTACTACCCCGACAAGGTGTTCAGA
TCTAGCGTGCTGCACAGCACCCAGGACCTGTTTCTGCCCTTCTTCAGCAACGTGACCTGGTTCCA
CGCCATCCATGTGTCCGGCACCAATGGCACCAAGAGATTCGACAACCCCGTGCTGCCCTTCAACG
ATGGGGTGTACTTTGCCAGCACCGAGAAGTCCAACATCATCAGAGGCTGGATCTTCGGCACCACA
CTGGACAGCAAGACCCAGAGCCTGCTGATCGTGAACAACGCCACCAACGTGGTCATCAAAGTGTG
CGAGTTCCAGTTCTGCAACGACCCATTCCTGGGAGTCTACTACCACAAGAACAACAAGAGCTGGA
TGGAAAGCGAGTTCCGGGTGTACAGCAGCGCCAACAACTGCACCTTCGAGTACGTGTCCCAGCCT
TTCCTGATGGACCTGGAAGGCAAGCAGGGCAACTTCAAGAACCTGCGCGAGTTCGTGTTCAAGAA
CATCGACGGCTACTTCAAGATCTACAGCAAGCACACCCCTATCAACCTCGTGCGGGATCTGCCTC
AGGGCTTTTCTGCTCTGGAACCTCTGGTGGACCTGCCTATCGGCATCAACATCACCCGGTTTCAG
ACCCTGCTGGCCCTGCACAGATCTTACCTGACACCTGGCGATAGCAGCTCTGGATGGACAGCTGG
CGCCGCTGCCTATTATGTGGGCTACCTGCAGCCTCGGACCTTCCTGCTGAAGTACAACGAGAACG
GCACCATCACCGACGCCGTGGATTGTGCTCTGGATCCCCTGAGCGAGACAAAGTGTACCCTGAAG
TCCTTCACCGTGGAAAAGGGCATCTACCAGACCAGCAACTTCAGAGTGCAGCCCACCGAGAGCAT
CGTGCGGTTCCCCAATATCACCAATCTGTGCCCCTTCGGCGAGGTGTTCAATGCCACAAGATTTG
CCAGCGTGTACGCCTGGAACCGGAAGAGAATCAGCAACTGCGTGGCCGACTACAGCGTGCTGTAC
AATAGCGCCAGCTTCAGCACCTTCAAGTGCTACGGCGTGTCACCCACCAAGCTGAACGACCTGTG
CTTCACCAATGTGTACGCCGACAGCTTCGTGATCAGAGGCGACGAAGTTCGGCAGATCGCTCCTG
GACAGACAGGCAAGATCGCCGATTACAACTACAAGCTGCCCGACGACTTCACCGGCTGCGTGATC
GCCTGGAATAGCAACAACCTGGACTCCAAAGTCGGCGGCAACTACAACTACCTGTACCGGCTGTT
CCGGAAGTCCAATCTGAAGCCCTTCGAGCGGGACATCTCCACCGAAATCTATCAGGCCGGCAGCA
CCCCTTGTAACGGCGTGGAAGGCTTCAACTGCTACTTCCCACTGCAGTCCTACGGCTTTCAGCCT
ACCAATGGCGTGGGCTATCAGCCCTATAGAGTGGTGGTGCTGAGCTTCGAACTGCTGCATGCCCC
TGCTACCGTGTGCGGCCCTAAGAAGTCTACCAACCTGGTCAAGAACAAATGCGTGAACTTCAACT
TCAACGGCCTGACCGGCACAGGCGTGCTGACAGAGAGCAACAAGAAGTTCCTGCCTTTCCAGCAG
TTTGGCCGGGATATCGCCGATACCACAGACGCCGTTAGAGATCCCCAGACACTGGAAATCCTGGA
CATCACCCCATGCAGCTTTGGCGGCGTGTCCGTGATCACACCTGGCACCAATACCAGCAATCAGG
TGGCCGTGCTGTATCAGGACGTGAACTGTACAGAGGTGCCAGTGGCCATTCACGCCGATCAGCTG
ACACCCACTTGGAGAGTGTACTCCACCGGCTCCAACGTGTTCCAGACTAGAGCCGGATGTCTGAT
CGGAGCCGAGCATGTGAACAACAGCTACGAGTGCGACATCCCCATCGGAGCTGGCATCTGTGCCA
GCTACCAGACACAGACAAATAGCCCCAGACGGGCCAGAAGCGTGGCCTCTCAGAGCATCATTGCC
TACACAATGAGCCTGGGCGCCGAGAATTCTGTGGCCTACAGCAACAACTCTATCGCTATCCCCAC
CAACTTCACCATCAGCGTGACCACCGAGATCCTGCCTGTGTCCATGACCAAGACCAGCGTGGACT
GCACCATGTACATCTGCGGCGATTCCACCGAGTGCAGCAACCTGCTGCTGCAGTACGGCAGCTTC
TGCACCCAGCTGAATAGAGCCCTGACAGGGATCGCCGTGGAACAGGACAAGAACACCCAAGAGGT
GTTCGCCCAAGTGAAGCAGATCTACAAGACCCCTCCTATCAAGGACTTCGGCGGCTTCAATTTCA
GCCAGATTCTGCCCGATCCTAGCAAGCCCAGCAAGCGGAGCTTTATCGAGGACCTGCTGTTCAAC
AAAGTGACACTGGCCGACGCCGGCTTCATCAAGCAGTATGGCGATTGCCTGGGCGACATTGCCGC
CAGAGATCTGATTTGCGCCCAGAAGTTTAACGGACTGACAGTGCTGCCTCCTCTGCTGACCGATG
AGATGATCGCCCAGTACACATCTGCTCTGCTGGCCGGCACAATCACCAGCGGATGGACATTTGGA
GCTGGCGCAGCCCTGCAGATCCCCTTTGCTATGCAGATGGCCTACCGGTTCAACGGCATCGGAGT
GACCCAGAATGTGCTGTACGAGAACCAGAAGCTGATCGCCAACCAGTTCAACAGCGCCATCGGCA
AGATCCAGGATAGCCTGTCTAGCACAGCCAGCGCTCTGGGCAAACTGCAGGACGTGGTCAATCAG
AACGCTCAGGCCCTGAACACCCTCGTGAAGCAGCTGAGCAGCAATTTCGGCGCCATCAGCTCCGT
GCTGAACGATATCCTGAGCCGGCTGGATAAGGTGGAAGCCGAGGTGCAGATCGACAGACTGATCA
CAGGCAGACTGCAGAGCCTCCAGACATACGTGACCCAGCAGCTGATCAGAGCCGCCGAGATTAGA
GCCTCTGCCAATCTGGCCGCCACAAAGATGTCTGAGTGTGTGCTGGGCCAGAGCAAGAGAGTGGA
TTTCTGCGGCAAGGGCTACCACCTGATGAGCTTTCCACAGTCTGCTCCTCACGGCGTGGTGTTTC
TGCATGTGACCTACGTGCCCGCTCAAGAGAAGAACTTCACAACAGCCCCTGCCATCTGCCACGAC
GGAAAGGCCCATTTTCCTAGAGAAGGCGTGTTCGTCAGCAACGGCACCCATTGGTTCGTGACACA
GCGGAACTTCTACGAGCCCCAGATCATCACCACCGACAACACCTTCGTGTCTGGCAACTGTGACG
TCGTGATCGGCATTGTGAACAATACCGTGTACGACCCTCTGCAGCCCGAGCTGGACAGCTTCAAA
GAGGAACTGGACAAGTACTTTAAGAACCACACAAGCCCCGACGTGGACCTGGGCGATATTAGCGG
CATCAATGCCAGCGTCGTGAACATCCAGAAAGAGATCGACCGGCTGAACGAGGTGGCCAAGAATC
TGAACGAGAGCCTGATCGACCTGCAAGAACTGGGGAAGTACGAGCAGTACATCAAGTGGCCCTGG
TACATCTGGCTGGGCTTTATCGCCGGACTGATTGCCATCGTGATGGTCACAATCATGCTGTGCTG
CATGACCAGCTGCTGTAGCTGCCTGAAGGGCTGTTGCAGCTGTGGCAGCTGCTGCAAGTTCGACG
AGGATGATAGCGAGCCTGTGCTGAAGGGCGTGAAACTGCACTACTAATGA
1-66 underlined nucleotide residues represent DNA sequence (nucleotide sequence)
of t-PA leader sequence and 67-3885 nucleotide residues represent DNA sequence
(nucleotide sequence) of S gene.
SEQ ID NO.: 7-Amino acid sequence of S1 region of S gene with IgE leader sequence
MDWTWILFLVAAATRVHSFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTV
EKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRAR
1-18 underlined amino acid residues represent amino acid sequence of IgE leader
sequence and 19-702 ammo acid residues represent amino acid sequence of full-
length S1 region of S protein.
SEQ ID NO.: 8-Nucleotide sequence of SI region of S gene with IgE leader sequence
ATGGATTGGACCTGGATTCTGTTTCTGGTGGCCGCTGCCACAAGAGTGCATAGCTTCGTCTTTCT
CGTGCTGCTGCCTCTGGTGTCCAGCCAGTGTGTGAACCTGACCACAAGAACACAGCTGCCTCCAG
CCTACACCAACAGCTTTACCAGAGGCGTGTACTACCCCGACAAGGTGTTCAGATCTAGCGTGCTG
CACAGCACCCAGGACCTGTTTCTGCCCTTCTTCAGCAACGTGACCTGGTTCCACGCCATCCATGT
GTCCGGCACCAATGGCACCAAGAGATTCGACAACCCCGTGCTGCCCTTCAACGATGGGGTGTACT
TTGCCAGCACCGAGAAGTCCAACATCATCAGAGGCTGGATCTTCGGCACCACACTGGACAGCAAG
ACCCAGAGCCTGCTGATCGTGAACAACGCCACCAACGTGGTCATCAAAGTGTGCGAGTTCCAGTT
CTGCAACGACCCCTTCCTGGGCGTCTACTACCACAAAAACAACAAGAGCTGGATGGAAAGCGAGT
TCCGGGTGTACAGCAGCGCCAACAACTGCACCTTCGAGTACGTGTCCCAGCCTTTCCTGATGGAT
CTGGAAGGCAAGCAGGGCAACTTCAAGAACCTGCGCGAGTTCGTGTTCAAGAACATCGACGGCTA
CTTCAAGATCTACAGCAAGCACACCCCTATCAACCTCGTGCGGGATCTGCCTCAGGGCTTTTCTG
CTCTGGAACCTCTGGTGGACCTGCCTATCGGCATCAACATCACCCGGTTTCAGACCCTGCTGGCC
CTGCACAGATCTTACCTGACACCTGGCGATAGCAGCTCTGGATGGACAGCTGGCGCCGCTGCCTA
TTATGTGGGCTACCTGCAGCCTCGGACCTTCCTGCTGAAGTACAACGAGAACGGCACCATCACCG
ACGCCGTGGATTGTGCTCTGGATCCCCTGAGCGAGACAAAGTGTACCCTGAAGTCCTTCACCGTG
GAAAAGGGCATCTACCAGACCAGCAACTTCAGAGTGCAGCCCACCGAGAGCATCGTGCGGTTCCC
CAATATCACCAATCTGTGCCCCTTCGGCGAGGTGTTCAATGCCACCAGATTTGCCAGCGTGTACG
CCTGGAACCGGAAGAGAATCAGCAACTGCGTGGCCGACTACAGCGTGCTGTACAATAGCGCCAGC
TTCAGCACCTTCAAGTGCTACGGCGTGTCCCCTACCAAGCTGAACGACCTGTGCTTCACCAATGT
GTACGCCGACAGCTTCGTGATCAGAGGCGACGAAGTTCGGCAGATCGCTCCTGGACAGACAGGCA
AGATCGCCGATTACAACTACAAGCTGCCCGACGACTTCACCGGCTGCGTGATCGCCTGGAATAGC
AACAACCTGGACTCCAAAGTCGGCGGCAACTACAACTACCTGTACCGGCTGTTCCGGAAGTCCAA
TCTGAAGCCCTTCGAGCGGGACATCTCCACCGAAATCTATCAGGCCGGCAGCACCCCTTGTAACG
GCGTGGAAGGCTTCAACTGCTACTTCCCACTGCAGTCCTACGGCTTTCAGCCTACCAATGGCGTG
GGCTATCAGCCCTATAGAGTGGTGGTGCTGAGCTTCGAACTGCTGCATGCCCCTGCTACCGTGTG
CGGCCCTAAGAAGTCTACCAACCTGGTCAAGAACAAATGCGTGAACTTCAACTTCAACGGCCTGA
CCGGCACAGGCGTGCTGACAGAGAGCAACAAGAAGTTCCTGCCTTTCCAGCAGTTTGGCCGGGAT
ATCGCCGATACCACAGACGCCGTTAGAGATCCCCAGACACTGGAAATCCTGGACATCACCCCATG
CAGCTTTGGCGGAGTGTCTGTGATCACCCCTGGCACCAATACCAGCAATCAGGTGGCCGTGCTGT
ATCAGGACGTGAACTGTACAGAGGTGCCAGTGGCCATTCACGCCGATCAGCTGACACCCACTTGG
AGAGTGTACTCCACCGGCTCCAACGTGTTCCAGACTAGAGCCGGATGTCTGATCGGAGCCGAGCA
TGTGAACAACAGCTACGAGTGCGACATCCCCATCGGAGCTGGCATCTGTGCCAGCTACCAGACAC
AGACAAACAGCCCCAGACGGGCCAGATAATGA
1-54 nucleotide residues represent DNA sequence (nucleotide sequence) of IgE
leader sequence and 55-2112 nucleotide residues represent DNA sequence (nucleotide
sequence) of S1 region of S gene.
SEQ ID NO.: 9-Amino acid sequence of S1 region of S gene with t-PA leader sequence
MDAMKRGLCCVLLLCGAVFVSPFVFLVLLPLVSSQCVNLTTRTQLPPAYTNSFTRGVYYPDKVFR
SSVLHSTQDLFLPFFSNVTWFHAIHVSGTNGTKRFDNPVLPFNDGVYFASTEKSNIIRGWIFGTT
LDSKTQSLLIVNNATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKCTLK
SFTVEKGIYQTSNFRVQPTESIVRFPNITNLCPFGEVFNATRFASVYAWNRKRISNCVADYSVLY
NSASFSTFKCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFPLQSYGFQP
TNGVGYQPYRVVVLSFELLHAPATVCGPKKSTNLVKNKCVNFNFNGLTGTGVLTESNKKFLPFQQ
FGRDIADTTDAVRDPQTLEILDITPCSFGGVSVITPGTNTSNQVAVLYQDVNCTEVPVAIHADQL
TPTWRVYSTGSNVFQTRAGCLIGAEHVNNSYECDIPIGAGICASYQTQTNSPRRAR
1-22 underlined amino acid residues represent amino acid sequence of t-PA leader
sequence and 23-706 amino acid residues represent amino acid sequence of full-
length S1 region of S protein,
SEQ ID NO.: 10-Nucleotide sequence of S1 region of S gene with t-PA leader sequence
ATGGATGCTATGAAGCGAGGACTGTGCTGCGTGCTGCTGCTGTGTGGTGCAGTGTTCGTGTCCCC
TTTCGTGTTCCTGGTCCTGCTGCCTCTGGTGTCCAGCCAGTGTGTGAACCTGACCACCAGAACAC
AGCTGCCTCCAGCCTACACCAATAGCTTCACCAGGGGCGTGTACTACCCCGACAAGGTGTTCAGA
TCTAGCGTGCTGCACAGCACCCAGGACCTGTTTCTGCCCTTCTTCAGCAACGTGACCTGGTTCCA
CGCCATCCATGTGTCCGGCACCAATGGCACCAAGAGATTCGACAACCCCGTGCTGCCCTTCAACG
ATGGGGTGTACTTTGCCAGCACCGAGAAGTCCAACATCATCAGAGGCTGGATCTTCGGCACCACA
CTGGACAGCAAGACCCAGAGCCTGCTGATCGTGAACAACGCCACCAACGTGGTCATCAAAGTGTG
CGAGTTCCAGTTCTGCAACGACCCATTCCTGGGAGTCTACTACCACAAGAACAACAAGAGCTGGA
TGGAAAGCGAGTTCCGGGTGTACAGCAGCGCCAACAACTGCACCTTCGAGTACGTGTCCCAGCCT
TTCCTGATGGACCTGGAAGGCAAGCAGGGCAACTTCAAGAACCTGCGCGAGTTCGTGTTCAAGAA
CATCGACGGCTACTTCAAGATCTACAGCAAGCACACCCCTATCAACCTCGTGCGGGATCTGCCTC
AGGGCTTTTCTGCTCTGGAACCTCTGGTGGACCTGCCTATCGGCATCAACATCACCCGGTTTCAG
ACCCTGCTGGCCCTGCACAGATCTTACCTGACACCTGGCGATAGCAGCTCTGGATGGACAGCTGG
CGCCGCTGCCTATTATGTGGGCTACCTGCAGCCTCGGACCTTCCTGCTGAAGTACAACGAGAACG
GCACCATCACCGACGCCGTGGATTGTGCTCTGGATCCCCTGAGCGAGACAAAGTGTACCCTGAAG
TCCTTCACCGTGGAAAAGGGCATCTACCAGACCAGCAACTTCAGAGTGCAGCCCACCGAGAGCAT
CGTGCGGTTCCCCAATATCACCAATCTGTGCCCCTTCGGCGAGGTGTTCAATGCCACAAGATTTG
CCAGCGTGTACGCCTGGAACCGGAAGAGAATCAGCAACTGCGTGGCCGACTACAGCGTGCTGTAC
AATAGCGCCAGCTTCAGCACCTTCAAGTGCTACGGCGTGTCACCCACCAAGCTGAACGACCTGTG
CTTCACCAATGTGTACGCCGACAGCTTCGTGATCAGAGGCGACGAAGTTCGGCAGATCGCTCCTG
GACAGACAGGCAAGATCGCCGATTACAACTACAAGCTGCCCGACGACTTCACCGGCTGCGTGATC
GCCTGGAATAGCAACAACCTGGACTCCAAAGTCGGCGGCAACTACAACTACCTGTACCGGCTGTT
CCGGAAGTCCAATCTGAAGCCCTTCGAGCGGGACATCTCCACCGAAATCTATCAGGCCGGCAGCA
CCCCTTGTAACGGCGTGGAAGGCTTCAACTGCTACTTCCCACTGCAGTCCTACGGCTTTCAGCCT
ACCAATGGCGTGGGCTATCAGCCCTATAGAGTGGTGGTGCTGAGCTTCGAACTGCTGCATGCCCC
TGCTACCGTGTGCGGCCCTAAGAAGTCTACCAACCTGGTCAAGAACAAATGCGTGAACTTCAACT
TCAACGGCCTGACCGGCACAGGCGTGCTGACAGAGAGCAACAAGAAGTTCCTGCCTTTCCAGCAG
TTTGGCCGGGATATCGCCGATACCACAGACGCCGTTAGAGATCCCCAGACACTGGAAATCCTGGA
CATCACCCCATGCAGCTTTGGCGGCGTGTCCGTGATCACACCTGGCACCAATACCAGCAATCAGG
TGGCCGTGCTGTATCAGGACGTGAACTGTACAGAGGTGCCAGTGGCCATTCACGCCGATCAGCTG
ACACCCACTTGGAGAGTGTACTCCACCGGCTCCAACGTGTTCCAGACTAGAGCCGGATGTCTGAT
CGGAGCCGAGCATGTGAACAACAGCTACGAGTGCGACATCCCCATCGGAGCTGGCATCTGTGCCA
GCTACCAGACACAGACAAACAGCCCCAGACGGGCCAGATAATGA
1-66 underlined nucleotide residues represent DNA sequence (nucleotide sequence)
of t-PA leader sequence and 67-2124 nucleotide residues represent DNA sequence
(nucleotide sequence) of S1 region of S gene.
SEQ ID NO.: 11-Amino acid sequence of full-length S gene (Hexapro) with IgE
leader sequence
MDWTWILFLVAAATRVHSFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTV
EKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSIIAYTMS
LGAENSVAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSPIEDLLFNKVTL
ADAGFIKQYGDCLGDIAARDLICAQKFNGLTVLPPLLTDEMIAQYTSALLAGTITSGWTFGAGPA
LQIPFPMQMAYRFNGIGVTQNVLYENQKLIANQFNSAIGKIQDSLSSTPSALGKLQDVVNQNAQA
LNTLVKQLSSNFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKWPWYIWL
GFIAGLIAIVMVTIMLCCMTSCCSCLKGCCSCGSCCKFDEDDSEPVLKGVKLHY
1-18 underlined amino acid residues represent amino acid sequence of IgE leader
sequence and 19-1289 amino acid residues represent amino acid sequence of full-
length S protein. Further underlined proline residues in 19-1289 region represent
six proline substitutions (K986P, V987P, F817P, A892P, A899P, and A942P) which is
referred herein as Hexapro substitution.
SEQ ID NO.: 12-Nucleotide sequence of full-length S gene (Hexapro) with IgE leader
sequence
ATGGATTGGACCTGGATTCTGTTTCTGGTGGCCGCTGCCACAAGAGTGCACAGCTTTGTCTTTCT
CGTGCTGCTGCCTCTGGTGTCCAGCCAGTGTGTGAACCTGACCACAAGAACACAGCTGCCTCCAG
CCTACACCAACAGCTTTACCAGAGGCGTGTACTACCCCGACAAGGTGTTCAGATCTAGCGTGCTG
CACAGCACCCAGGACCTGTTTCTGCCCTTCTTCAGCAACGTGACCTGGTTCCACGCCATCCACGT
GTCCGGCACCAATGGCACCAAGAGATTCGACAACCCCGTGCTGCCCTTCAACGATGGGGTGTACT
TTGCCAGCACCGAGAAGTCCAACATCATCAGAGGCTGGATCTTCGGCACCACACTGGACAGCAAG
ACCCAGAGCCTGCTGATCGTGAACAACGCCACCAACGTGGTCATCAAAGTGTGCGAGTTCCAGTT
CTGCAACGACCCCTTCCTGGGCGTCTACTACCACAAAAACAACAAGAGCTGGATGGAAAGCGAGT
TCCGGGTGTACAGCAGCGCCAACAACTGCACCTTCGAGTACGTGTCCCAGCCTTTCCTGATGGAT
CTGGAAGGCAAGCAGGGCAACTTCAAGAACCTGCGCGAGTTCGTGTTCAAGAACATCGACGGCTA
CTTCAAGATCTACAGCAAGCACACCCCTATCAACCTCGTGCGGGATCTGCCTCAGGGCTTTTCTG
CTCTGGAACCTCTGGTGGACCTGCCTATCGGCATCAACATCACCCGGTTTCAGACCCTGCTGGCC
CTGCACAGATCTTACCTGACACCTGGCGATAGCAGCTCTGGATGGACAGCTGGCGCCGCTGCCTA
TTATGTGGGCTACCTGCAGCCTCGGACCTTCCTGCTGAAGTACAACGAGAACGGCACCATCACCG
ACGCCGTGGATTGTGCTCTGGATCCCCTGAGCGAGACAAAGTGCACCCTGAAGTCCTTCACCGTG
GAAAAGGGCATCTACCAGACCAGCAACTTCAGAGTGCAGCCCACCGAGAGCATCGTGCGGTTCCC
CAATATCACCAATCTGTGCCCCTTCGGCGAGGTGTTCAATGCCACCAGATTTGCCAGCGTGTACG
CCTGGAACCGGAAGAGAATCAGCAACTGCGTGGCCGACTACAGCGTGCTGTACAATAGCGCCAGC
TTCAGCACCTTCAAGTGCTACGGCGTGTCCCCTACCAAGCTGAACGACCTGTGCTTCACCAATGT
GTACGCCGACAGCTTCGTGATCAGAGGCGACGAAGTTCGGCAGATCGCTCCTGGACAGACAGGCA
AGATCGCCGATTACAACTACAAGCTGCCCGACGACTTCACCGGCTGCGTGATCGCCTGGAATAGC
AACAACCTGGACTCCAAAGTCGGCGGCAACTACAACTACCTGTACCGGCTGTTCCGGAAGTCCAA
TCTGAAGCCCTTCGAGCGGGACATCTCCACCGAAATCTATCAGGCCGGCAGCACCCCTTGTAACG
GCGTGGAAGGCTTCAACTGCTACTTCCCACTGCAGTCCTACGGCTTTCAGCCTACCAATGGCGTG
GGCTATCAGCCCTATAGAGTGGTGGTGCTGAGCTTCGAACTGCTGCATGCCCCTGCTACCGTGTG
CGGCCCTAAGAAGTCTACCAACCTGGTCAAGAACAAATGCGTGAACTTCAACTTCAACGGCCTGA
CCGGCACAGGCGTGCTGACAGAGAGCAACAAGAAGTTCCTGCCTTTCCAGCAGTTTGGCCGGGAT
ATCGCCGATACCACAGACGCCGTTAGAGATCCCCAGACACTGGAAATCCTGGACATCACCCCATG
CAGCTTTGGCGGAGTGTCTGTGATCACCCCTGGCACCAATACCAGCAATCAGGTGGCCGTGCTGT
ATCAGGACGTGAACTGTACAGAGGTGCCCGTGGCCATTCACGCCGATCAACTGACACCCACTTGG
AGAGTGTACTCCACCGGCTCCAACGTGTTCCAGACTAGAGCCGGATGTCTGATCGGAGCCGAGCA
CGTGAACAATAGCTACGAGTGCGACATCCCCATCGGCGCTGGCATCTGTGCCAGCTACCAGACAC
AGACAAATAGCCCCAGACGGGCCAGAAGCGTGGCCTCTCAGAGCATCATTGCCTACACAATGAGC
CTGGGCGCCGAGAATTCTGTGGCCTACAGCAACAACTCTATCGCTATCCCCACCAACTTCACCAT
CAGCGTGACCACCGAGATCCTGCCTGTGTCCATGACCAAGACCAGCGTGGACTGCACCATGTACA
TCTGCGGCGATTCCACCGAGTGCAGCAACCTGCTGCTGCAGTACGGCAGCTTCTGCACCCAGCTG
AATAGAGCCCTGACAGGGATCGCCGTGGAACAGGACAAGAATACCCAAGAGGTGTTCGCCCAAGT
GAAGCAGATCTACAAGACCCCTCCTATCAAGGACTTCGGCGGCTTCAATTTCAGCCAGATTCTGC
CCGATCCTAGCAAGCCCAGCAAGAGAAGCCCAATCGAGGACCTGCTGTTCAACAAAGTGACACTG
GCCGACGCCGGCTTCATCAAGCAGTATGGCGATTGCCTGGGCGACATTGCCGCCAGAGATCTGAT
TTGCGCCCAGAAGTTTAACGGACTGACAGTGCTGCCTCCTCTGCTGACCGATGAGATGATCGCCC
AGTACACATCTGCTCTGCTGGCCGGCACAATCACCAGCGGATGGACATTTGGAGCAGGCCCAGCT
CTGCAGATCCCATTTCCAATGCAGATGGCCTACCGGTTCAACGGCATCGGAGTGACCCAGAATGT
GCTGTACGAGAACCAGAAGCTGATCGCCAACCAGTTCAACAGCGCCATCGGCAAGATCCAGGACA
GCCTGTCTAGCACACCTAGCGCTCTGGGCAAGCTGCAGGACGTGGTCAATCAGAACGCTCAGGCC
CTGAACACCCTCGTGAAGCAGCTGAGCAGCAATTTCGGCGCCATCAGCTCCGTGCTGAACGATAT
CCTGAGCAGACTGGATCCTCCAGAGGCCGAGGTGCAGATCGATAGACTGATCACAGGCCGGCTGC
AGTCCCTGCAGACATATGTGACACAGCAGCTGATCAGAGCCGCCGAGATTAGAGCCTCTGCCAAT
CTGGCCGCCACAAAGATGTCTGAGTGTGTGCTGGGCCAGAGCAAGAGAGTGGATTTCTGCGGCAA
GGGCTACCACCTGATGAGCTTTCCACAGTCTGCCCCTCACGGCGTGGTGTTTCTGCATGTGACAT
ACGTGCCCGCTCAAGAGAAGAACTTCACAACAGCCCCTGCCATCTGCCACGACGGAAAGGCCCAT
TTTCCTAGAGAAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGTGACGTCGTGATCGGCA
TTGTGAACAACACCGTGTACGACCCTCTGCAGCCCGAGCTGGACAGCTTCAAAGAGGAACTGGAC
AAGTACTTTAAGAACCACACAAGCCCCGACGTGGACCTGGGCGATATTAGCGGCATCAATGCCTC
CGTGGTCAACATCCAGAAAGAGATCGACCGGCTGAACGAGGTGGCCAAGAATCTGAACGAGAGCC
TGATCGACCTGCAAGAACTGGGGAAGTACGAGCAGTACATCAAGTGGCCCTGGTACATCTGGCTG
GGCTTTATCGCCGGACTGATTGCCATCGTGATGGTCACAATCATGCTGTGCTGCATGACCAGCTG
TTGCAGCTGCCTGAAGGGCTGCTGTAGCTGTGGCTCCTGCTGCAAGTTCGACGAGGATGATAGCG
AGCCTGTGCTGAAGGGCGTGAAACTGCACTACTAATGA
1-54 underlined nucleotide residues represent DNA sequence (nucleotide sequence)
of IgE leader sequence and 55-3873 nucleotide residues represent DNA sequence
(nucleotide sequence) of S gene (Hexapro).
SEQ ID NO.: 13-Amino acid sequence of full-length S gene (2P) with IgE leader
sequence
MDWTWILFLVAAATRVHSFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTV
EKGIYQTSNFRVQPTESIVRFPNITNLCPFGEVFNATRFASVYAWNRKRISNCVADYSVLYNSAS
FSTFKCYGVSPTKLNDLCFTNVYADSFVIRGDEVRQIAPGQTGKIADYNYKLPDDFTGCVIAWNS
NNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQDVNCTEVPVAIHADQLTPTW
RVYSTGSNVFQTRAGCLIGAEHVNNSYECDIPIGAGICASYQTQTNSPRRARSVASQSIIAYTMS
LGAENSVAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSFIEDLLFNKVTL
ADAGFIKQYGDCLGDIAARDLICAQKFNGLTVLPPLLTDEMIAQYTSALLAGTITSGWTFGAGAA
LQIPFAMQMAYRFNGIGVTQNVLYENQKLIANQFNSAIGKIQDSLSSTASALGKLQDVVNQNAQA
LNTLVKQLSSNFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKWPWYIWL
GFIAGLIAIVMVTIMLCCMTSCCSCLKGCCSCGSCCKFDEDDSEPVLKGVKLHY
1-18 underlined amino acid residues represent amino acid sequence of IgE leader
sequence and 19-1289 amino acid residues represent amino acid sequence of full-
length S protein. Further underlined proline residues in 19-1289 region represent
two proline substitutions (K986P, V987P) which is referred herein as 2P substitution.

### DEFINITIONS

The term “SARS-CoV-2”, “2019-nCoV” and “HCoV-19” as used herein refers to corona virus which outbreak in December 2019 and first reported in Wuhan, China.

The term “episome” as used herein refers to the plasmid DNA construct co-expressing the S gene or S1 region of S gene and the leader sequence that can undergo transcription and translation independently into host cell, preferably human muscle cells, skin cells or antigen presenting cells. The episome should go into the host cell nucleus and express the target protein, preferably here S protein or S1 region of S protein using host cell machinery and without integrating into the host cell genome.

The term “signal peptide” as described herein is a peptide (sometimes referred to as signal sequence, targeting signal, localization signal, localization sequence, transit peptide, leader sequence or leader peptide) is a short peptide (usually 16-30 amino acids long) present at the N-terminus of the newly synthesized proteins that are destined towards the secretory pathway.

The terms “polypeptide”, “protein” and “amino acid sequence” as used herein generally refer to a polymer of amino acid residues and are not limited to a minimum length of the product. Thus, peptides, oligopeptides, dimers, multimers, and the like, are included within the definition. Both full-length proteins and fragments thereof are encompassed by the definition.

The term “nucleotide” as used herein generally refer to a sequence of nucleic acid residues and are not limited to a minimum length of the product. Both full-length nucleotide and fragments or variants thereof are encompassed by the definition.

The term “fragment” or “variant” as used herein refers to a functional part of a full-length polypeptide, protein or nucleotide, whose sequence is not identical to the respective full-length polypeptide, protein or nucleotide but retains the same function as the full-length polypeptide, protein or nucleotide. The said functional fragment or the functional variant may have more, less, or the same number of residues than the corresponding native molecule and/or may contain one or more amino acid or nucleotide substitutions.

An “immunogenic composition”, “immunogenic formulation” and “formulation” are used interchangeably and refer to a composition or formulation that comprises an antigenic molecule where administration of the composition to a subject results in the development of a humoral and/or a cellular immune response to the antigenic molecule of interest in the subject. The immunogenic composition can be introduced directly into a recipient subject, such as by injection, inhalation, oral, intranasal or any other parenteral, mucosal or transdermal (e.g., intra-rectally or intra-vaginally) route of administration.

The term “pseudovirus” as described herein is a synthetic or recombinant virus with its core and envelope proteins derived from different virus. Example measles virus expressing SARS S protein. The term “pseudovirion” has the same meaning as “pseudovirus” term, but it is commonly used with neutralizing antibodies assay. The terms “polypeptide”, “protein” and “amino acid sequence” as used herein generally refer to a polymer of amino acid residues and are not limited to a minimum length of the product. Thus, peptides, oligopeptides, dimers, multimers, and the like, are included within the definition. Both full-length proteins and fragments thereof are encompassed by the definition.

The term “pharmaceutical formulation” refers to preparations, which are in such form as to permit the biological activity of the active ingredients to be unequivocally effective. The term “pharmaceutical formulation”, “pharmaceutical composition” and “composition” can be used here interchangeably.

The term “excipient” refers to an agent that may be added to a formulation to stabilize the active drug substance in the formulated form to adjust and maintain osmolality and pH of the pharmaceutical preparations. Examples of commonly used excipients include, but are not limited to, anesthetic compound, sugars, polyols, amino acids, surfactants and polymers. “Pharmaceutically acceptable” excipients are those which can reasonably be administered to a subject mammal to provide an effective dose of the active ingredient employed.

The term “treatment” or “therapeutics” as used herein, refers to any treatment of a disease in a mammal, particularly in a human. It includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

The terms “patient” and “subject” are used interchangeably and are used in their conventional sense to refer to a living organism suffering from or prone to a condition that can be prevented or treated by administration of a composition of the present invention, and includes both humans and non-human animals. Examples of subjects include, but are not limited to, humans, chimpanzees and other apes and monkey species; farm animals such as cattle, sheep, pigs, goats and horses: domestic mammals such as dogs and cats: laboratory animals including rodents such as mice, rats and guinea pigs; birds, including domestic, wild and game birds such as chickens, turkeys and other gallinaceous birds, ducks, geese, and the like. The term does not denote a particular age. Thus, adult, juvenile and new born individuals are of interest.

The terms “ZVTC_COV” and “VTC_COV” are meant to similar and used interchangeably.

The term “amino acid substitution” or “substitution” as used herein is the replacement of an amino acid at a particular position or location in a parent polypeptide sequence with another amino acid. For example, the substitution K986P refers to a variant polypeptide, in this case a variant of S protein of SARS-CoV-2, in which the lysine at position 986 is replaced with proline.

TABLE 1
Abbreviations of amino acid as used in the current application

| | Abbreviation | Abbreviation |
|---|---|---|
| Full Name | (3 Letter) | (1 Letter) |
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Abbreviations Used in the Current Application

γ: Gamma

2019-nCoV: novel coronavirus

A: Adenine

ACE2: Angiotensin-converting enzyme 2

APC: Antigen presenting cell

BGH: Bovine growth hormone

BSA: Bovine Serum albumin

C: Cytosine

CMV: Cytomegalovirus

CL: Cationic lipids

CoV: Coronavirus

CPE: Cytopathic effect

DNA: deoxyribonucleic acid

DMEM: Dulbecco's modified eagle medium

DOTMA: 1, 2-di-O-octadecenyl-3-trimethylammonium propane

DOTAP: N-[1-(2, 3-Dioleoyloxy) propyl]-N, N, N-trimethylammonium methyl-sulfate

ELISA: Enzyme-linked immunosorbent assay

ELISpot: Enzyme-linked immune absorbent spot

FACS: Fluorescence-activated cell sorting

FBS: Fetal bovine serum

FITC: Fluorescein isothiocyanate

G: Guanine

h: Hour

Hr: Hour

HRP: Horse radish peroxidase

ID: Intradermal

IFN: Interferon

IM: Intramuscular

IgG: Immunoglobulin G

IgE: Immunoglobulin E

MERS: Middle East respiratory syndrome coronavirus

MHC: Major histocompatibility complex

ml: milliliter

MNT: Micro-neutralization test

MV: Measles vector

%: Percentage

° C.: Degree Celsius

nM: nano molar

NFIS: Needle-free injection system

OD: Optical density

p: Plasmid

pDNA: plasmid DNA

PBS: Phosphate buffer saline

RBD: Receptor binding domain

RNA: Ribonucleic acid

RPMI: Roswell park memorial institute

S protein: Spike protein

SARS: Severe acute respiratory syndrome

SC: Subcutaneous

T: Thymine

t-PA: Tissue plasminogen activator

TCID₅₀: 50% tissue culture infectious dose

TMB: 3, 3′, 5, 5′-tetramethlybenzidine

TNF: Tumor necrosis factor

VSV: Vesicular stomatitis virus

### Embodiments of the Invention

In one embodiment, the current invention provides a DNA construct which can be developed as a vaccine for the prophylaxis of SARS-CoV-2. In a preferred embodiment, the DNA construct according to the present invention comprising S gene of SARS-CoV-2 or a gene of S1 region of S protein of SARS-CoV-2.

In one of the embodiments, the DNA construct comprising a gene encoding S protein of SARS-CoV-2 or the truncated gene of S protein of SARS-CoV-2. The truncated gene of S protein of the present invention includes S1 region or a receptor binding domain RBD that binds to the human angiotensin converting Enzyme (ACE)-2 receptor.

In a preferred embodiment, the current invention provides DNA construct comprising a gene encoding S protein of SARS-CoV-2 has nucleotide sequence as set forth in SEQ ID NO.: 4 or SEQ ID NO.: 6.

In a preferred embodiment, the current invention provides amino acid sequence of DNA construct comprising a gene encoding S protein of SARS-CoV-2 wherein said amino acid sequence is SEQ ID NO.: 3 or SEQ ID NO.: 5.

In one of the preferred embodiments, the current invention provides DNA construct comprising a gene encoding S protein of SARS-CoV-2 has nucleotide sequence from nucleotide residues from 55 to 3873 of SEQ ID NO.: 4 or its fragment or its variant thereof.

In one of the preferred embodiments, the current invention provides DNA construct comprising a gene encoding S protein of SARS-CoV-2 has nucleotide sequence from nucleotide residues from 67 to 3885 of SEQ ID NO.: 6 or its fragment or its variant thereof.

In one of the embodiments, the current invention provides DNA construct comprising a gene encoding prefusion stabilized S protein of SARS-CoV-2 with substitution, wherein S protein of SARS-CoV-2 has substitution selected from K986P, V987P, F817P, A892P, A899P and A942P. In one of such preferred embodiments, the current invention provides DNA construct comprising a gene encoding prefusion stabilized S protein of SARS-CoV-2 with substitution, wherein S protein of SARS-CoV-2 has substitution selected from K986P and V987P substitutions. In one of such preferred embodiments, the current invention provides DNA construct comprising a gene encoding prefusion stabilized S protein of SARS-CoV-2 with substitution, wherein S protein of SARS-CoV-2 has substitution K986P, V987P, F817P, A892P, A899P and A942P substitutions.

The DNA construct comprising a gene encoding S protein of SARS-CoV-2 with K986P, V987P, F817P, A892P, A899P and A942P substitutions according to the present invention has nucleotide sequence as set forth in SEQ ID NO.: 12. In one of the preferred embodiments, DNA construct comprising a gene encoding S protein of SARS-CoV-2 with K986P, V987P, F817P, A892P, A899P and A942P substitutions according to the present invention has nucleotide sequence from nucleotide residues from 55 to 3873 of SEQ ID NO.: 12 or its fragment or its variant thereof.

In a preferred embodiment, the current invention provides DNA construct comprising a gene encoding truncated gene of S protein of SARS-CoV-2 (S1 protein) has nucleotide sequence as set forth in SEQ ID NO.: 8 or SEQ ID NO.: 10. In a preferred embodiment, the current invention provides amino acid sequence of DNA construct comprising a gene encoding truncated gene of S protein of SARS-CoV-2 (S1 protein) wherein said amino acid sequence is SEQ ID NO.: 7 or SEQ ID NO.: 9.

In one of the preferred embodiments, the current invention provides DNA construct comprising a gene encoding truncated gene of S protein of SARS-CoV-2 (S1 protein) has nucleotide sequence from nucleotide residues from 55 to 2112 of SEQ ID NO.: 8 or its fragment or its variant thereof.

In one of the preferred embodiments, the current invention provides DNA construct comprising a gene encoding truncated gene of S protein of SARS-CoV-2 (S1 protein) has nucleotide sequence from nucleotide residues from 67 to 2124 of SEQ ID NO.: 10 or its fragment or its variant thereof.

In one of the preferred embodiments, the current invention provides DNA construct comprising a gene encoding S protein of SARS-CoV-2 has nucleotide sequence having at least 95% identity over an entire length of the nucleotide sequence as set forth in SEQ ID NO.: 4 or SEQ ID NO.: 6.

In one of the preferred embodiments, the current invention provides DNA construct comprising a gene encoding S protein of SARS-CoV-2 has nucleotide sequence having at least 95% identity over an entire length of the nucleotide sequence as set forth in SEQ ID NO.: 12.

In one of the preferred embodiments, the current invention provides DNA construct comprising a gene encoding truncated gene of S protein of SARS-CoV-2 (S1 protein) has nucleotide sequence having at least 95% identity over an entire length of the nucleotide sequence as set forth in SEQ ID NO.: 8 or SEQ ID NO.: 10.

In one of the embodiments, the current invention provides DNA construct or its functional variant(s) thereof. Further, current invention also provides DNA construct or its functional variant(s) with optimized nucleotide gene sequence for the suitable host cell. Preferably, suitable host cell according to the present invention is E. coli.

In another embodiment, the present invention provides a vector comprising a gene encoding S protein of SARS-CoV-2 or a gene encoding S1 region of S protein of SARS-CoV-2. Any vector which can express target protein in vivo can be used according to the present invention. In a preferred embodiment, the vector according to the present invention is pVAX1. Other vectors such as pCDNA 3.1, pCDNA 4.0, pCMV, PCAGG etc. can also be used to express the target protein. The vector of the present invention may include human cytomegalovirus immediate-early (CMV) promoter for high-level expression in a wide range of mammalian cells, bovine growth hormone (BGH) polyadenylation signal for efficient transcription termination and polyadenylation of mRNA, kanamycin resistance gene for selection in E. coli or suitable combination thereof.

In certain embodiment, the present invention provides a vector comprising a gene encoding S protein of SARS-CoV-2 or a gene encoding S1 region of S protein of SARS-CoV-2 and a gene encoding signal peptide. In a preferred embodiment, the signal peptide is IgE signal peptide or t-PA signal peptide.

In one of the embodiments, the present invention provides a method of preparing vector comprising a gene encoding S protein of SARS-CoV or a gene encoding S1 region of S protein of SARS-CoV-2 optionally with a gene encoding signal peptide. In a preferred embodiment, the signal peptide is IgE signal peptide or t-PA signal peptide.

In another embodiment, the present invention provides a vector comprising functional variant(s) of a gene encoding S protein of SARS-CoV-2 or a gene encoding S1 region of S protein of SARS-CoV-2 and optionally with a gene encoding signal peptide.

In another embodiment, the vector prepared according to the present invention further comprises regulatory element(s) required for the expression of S gene of SARS-CoV-2 or S1 region of S gene of SARS-CoV-2.

In one more embodiment, the present invention provides a method of administration of DNA construct comprising S gene of SARS-CoV-2 or S1 region of S gene of SARS-CoV-2 into a subject. The DNA construct may further comprise a gene encoding IgE signal peptide or a gene encoding t-PA signal peptide. In a preferred embodiment, the present invention provides a method of administration of a vector comprising a gene encoding S protein of SARS-CoV-2 or a gene encoding S1 region of S protein of SARS-CoV-2 optionally with a gene encoding signal peptide. Signal peptide according to the present invention can be IgE signal peptide or t-PA signal peptide.

In another embodiment of the present invention, the plasmid DNA (pDNA) vector co-expressing the S gene and the signal peptide is transformed into suitable E. coli host cell for large scale production of plasmid DNA for immunization.

In another embodiment of the present invention, scalable production process using batch and fed-batch method can be used with suitable different media compositions comprising of yeast extract, tryptone, glycerol and other suitable ingredients available for high density E. coli culture. Also, temperature range from 30° C. to 42° C. can be used according to the present invention to increase plasmid yield from bacterial biomass.

In one of the embodiments of the present invention, purification process comprising one or more of the following steps: (a) lysis of host cell containing plasmid DNA; (b) clarifying the lysate by filtration to obtain clarified lysate; (c) treating lysate to remove endotoxin and other impurities; (d) purifying the treated solution of step (c) with plasmid DNA using one or more of the chromatography techniques selected from affinity chromatography (AC), ion exchange chromatography (IEC) and/or hydrophobic interaction chromatography (HIC); (e) concentrating the purified plasmid comprising of one or more following steps of (i) precipitation, (ii) diafiltration and/or (iii) lyophilization.

In another embodiment, the present invention provides a method of making an immunogenic composition of the plasmid DNA vector comprising a gene encoding S protein of SARS-CoV-2 or a gene encoding S1 region of S protein of SARS-CoV-2 with a gene encoding leader sequence. The immunogenic composition of the present invention can be prepared in water or saline. The immunogenic composition preferably comprises buffer, stabilizer, adjuvant and optionally other suitable pharmaceutical excipient(s). In a preferred embodiment of the present invention, the immunogenic composition comprises of (a) buffer preferably phosphate buffered saline (PBS); (b) stabilizer(s) selected from free radical scavenger and/or metal ions chelators; (c) other pharmaceutical excipient(s) selected from bupivacaine hydrochloride and/or sugars selected from Vi-polysaccharide, zymogen and/or chitosan; and (d) adjuvant(s) selected from aluminum hydroxide gel, bacterially derived adjuvant, lipophilic adjuvant, hydrophilic adjuvant, Complete Freund's adjuvant (CFA), Incomplete Freund's adjuvant (IFA), mono phosphoryl lipid A, beta-sitosterol and suitable combination thereof.

In one of the preferred embodiments, the immunogenic composition or formulation of the present invention is a liquid formulation comprising buffer and DNA plasmid construct with spike protein gene region from SARS-CoV-2 virus. The preferred buffer according to the current invention is phosphate buffered saline.

In one of the preferred embodiments, the immunogenic composition or formulation of the present invention is a liquid formulation comprising buffer and DNA plasmid construct with S1 region of spike protein gene region from SARS-CoV-2 virus. The preferred buffer according to the current invention is phosphate buffered saline.

In one of the preferred embodiments, the immunogenic composition or formulation of the present invention is liposomal formulation. In one of such embodiments, lipid entrapment or complexation methods using cationic lipids (CL) comprising of one or more lipids selected from (a) DOTMA (1, 2-di-O-octadecenyl-3-trimethylammonium propane) and (b) DOTAP (N-[1-(2, 3-Dioleoyloxy) propyl]-N, N, N-trimethylammonium methyl-sulfate) for plasmid DNA delivery can be used. In one of the embodiments, molar ratio of cationic lipid nitrogen (N) to pDNA phosphate (P) of the formulation of the present invention is selected from 1, 2 and 3. In one of the embodiments, molar ratio of the cationic lipid to the helper lipid of the formulation is 1:1 for the formulations which contain helper lipid(s).

In certain embodiment, the pDNA liposome formulation prepared according to the current invention is comprised of single vial formulation or two-vial formulation. Single vial formulation can be liquid injection or lyophilized powder for injection. Two-vial based formulation includes one vial containing pDNA and the other vial containing lipid dispersion. Both the vials can be mixed at the time of administration.

The DNA vaccine comprising immunogenic composition or formulation prepared according to the present invention is stable at least for 6 months at 5±3° C. and at stable for 3 months at 25±2° C.

In another embodiment, the DNA construct or the vector of the present invention is injected into a subject intramuscularly or intradermally. The immunization method according to the present invention includes anyone of the needle-free injection system (NFIS) or electroporator or direct needle injection.

In one of the embodiments, the present invention provides an immunogenic composition comprising DNA construct or the vector of the present invention. In further embodiment, the present invention provides a method of making an immunogenic composition comprising DNA construct or the vector of the present invention.

In another embodiment, the present invention provides an immunogenic composition comprising DNA construct or its functional variant(s).

In one of the embodiments, the present invention provides a vaccine comprising DNA construct or the vector of the present invention. In a preferred embodiment, the present invention provides a DNA vaccine comprising S gene of SARS-CoV-2 or S1 region of S gene of SARS-CoV-2. In further embodiment, the present invention provides a DNA vaccine comprising S gene of SARS-CoV-2 or S1 region of S gene of SARS-CoV-2 and a gene encoding signal peptide selected from IgE signal peptide or t-PA signal peptide.

In a preferred embodiment, the vaccine according to the present invention includes a vector comprising a gene encoding S protein of SARS-CoV-2 or a vector comprising a gene encoding S1 region of S protein of SARS-CoV-2. In a further embodiment, the vaccine according to the present invention provides a vector comprising a gene encoding S protein of SARS-CoV-2 or a gene encoding S1 region of S protein of SARS-CoV-2 and a gene encoding signal peptide selected from IgE signal peptide or t-PA signal peptide.

In one of the embodiments, the vaccine prepared according to the current invention induces humoral and/or cellular immune response into the subject. The cellular response can be measured by ELISA or FACS or ELISpot. In one of the embodiments, the vaccine prepared according to the current invention induces generation of anti-viral CD8⁺ T cell responses. In one of the embodiments, the vaccine prepared according to the current invention induces generation of anti-viral CD4⁺ T cell responses. In one of the embodiments, the vaccine prepared according to the current invention induces IFN-γ expression.

In another embodiment, the vaccine prepared according to the current invention induces generation of coronavirus neutralizing antibodies into the subject.

In another embodiment, the present invention provides method of treating or method of preventing coronavirus or its related disease by administration of suitable therapeutic dose of the DNA vaccine prepared according to the current invention.

In one of the embodiments, the DNA vaccine was found to be well tolerated and no any apparent signs of toxicity at repeated absolute human dose (6 mg). In one of the embodiments, the DNA vaccine of the present invention is co-delivered with cytokines to enhance the production of a Th1 immune response beneficial in a viral infection.

### DETAILED DESCRIPTION OF THE INVENTION

The current invention provides a DNA construct comprising S gene of SARS-CoV-2. S gene as referred herein the current application can be full-length S gene or suitable truncated portion of S gene or functional variant(s) of S gene, preferably S1 region of S gene or suitable receptor binding domain containing portion of the S gene or suitable fragment of S gene which can induce immune response. In a preferred embodiment, the DNA construct according to the current invention comprising S gene of SARS-CoV-2. In a preferred embodiment, the DNA construct according to the current invention comprising S1 region of S gene of SARS-CoV-2. The DNA construct according to the current invention has nucleotide sequence as set forth in SEQ ID No. 4 or SEQ ID NO. 6 or SEQ ID NO. 8 or SEQ ID No. 10. The amino acid sequence expressed from the DNA construct prepared according to the current invention is selected from SEQ ID NO. 3, SEQ ID NO. 5, SEQ ID NO. 7 and SEQ ID NO. 9. Current invention also provides a DNA construct comprising S gene of SARS-CoV-2 that can provide higher expression of S gene. The said DNA construct comprising S gene of SARS-CoV-2 has ability to withstand heat stress, stable at room temperature, and stable upon multiple freeze-thaw cycles. The DNA construct that has ability to withstand heat stress, stable at room temperature, and stable upon multiple freeze-thaw cycles expresses an amino acid sequence of the prefusion stabilized S protein of SARS-CoV-2 with proline substitution. Said proline substitution according to the current invention is selected from K986P, V987P, F817P, A892P, A899P, A942P and suitable combination thereof. One of the preferred combinations of proline substitution is K986P and V987P. It can be referred as 2P (two proline substitutions). Another preferred combination of proline substitution is K986P, V987P, F817P, A892P, A899P and A942P. It can be referred as hexaPro (six proline substitutions). The DNA construct encoding S protein of SARS-CoV-2 with six proline substitutions has nucleotide sequence as set forth in SEQ ID No. 12. The DNA construct encoding S protein of SARS-CoV-2 with two proline substitutions can be prepared by the codon optimized methods and vectors of the present invention as illustrated herein examples. The amino acid sequence expressed from the DNA construct that has ability to withstand heat stress, stable at room temperature, and stable upon multiple freeze-thaw cycles, according to the current invention is SEQ ID NO.: 11 (Hexapro) or SEQ ID NO.: 13 (2P). 2019-nCoV uses the densely glycosylated spike (S) protein to gain entry into host cells. The S protein is a trimeric class I fusion protein that exists in a metastable prefusion conformation that undergoes a substantial structural rearrangement to fuse the viral membrane with the host cell membrane. This process is triggered when the S1 subunit binds to a host cell receptor. Receptor binding destabilizes the prefusion trimer, resulting in shedding of the S1 subunit and transition of the S2 subunit to a stable postfusion conformation (3).

It is well known that 2019-nCoV S and SARS-CoV S share the same functional host cell receptor, ACE2. It is also reported that ACE2 bound to the 2019-nCoV S ectodomain with ˜15 nM affinity, which is ˜10- to 20-fold higher than ACE2 binding to SARS-CoV S. The high affinity of 2019-nCoV S for human ACE2 may contribute to the apparent ease with which 2019-nCoV can spread from human to human (3). Current invention provides the DNA construct encoding full-length S protein of SARS-CoV-2 or the DNA construct encoding S1 region of S protein of SARS-CoV-2.

DNA construct of the present invention includes construction of a vector carrying a gene encoding S protein of SARS-CoV-2. The vector of the current invention may carry SARS-CoV-2 antigen, a fragment thereof, a variant thereof or a combination thereof. The vector comprising DNA construct of the present invention can be plasmid DNA (pDNA). The vector according to the current invention may carry S1 region of S protein of SARS-CoV-2. The vector optionally may comprises a gene coding for IgE signal peptide. The signal peptide is involved in trafficking of the expressed S protein or the S1 protein to the cell membrane from where it is either secreted into the interstitial space or it can remain bound on the cell membrane where the S protein antigen or S1 region of S protein antigen is cross-presented to APCs. APCs by direct uptake of the antigen or by phagocytosis of antigen expressing somatic cell, present the antigen through their MHC I and MHC II complexes to CD4⁺ and CD8⁺ T cells. The secreted protein is also recognized by B cells via B cell receptors and presented through MHCII complex, inducing virus neutralizing.

Preferred vector according to the current invention is pVAX1 (Invitrogen, USA). The art of construction of the vector pVAX1 is well established and the vector has been widely used for construction of DNA vaccine (4 and 5). The vector of the present invention may include further regulatory element(s) required for the high level expression of full-length S protein or S1 region of S protein. Such regulatory elements and the vector comprising combination of regulatory elements are well disclosed in for e.g. patent documents WO2008085956, WO 2012046255 and WO 2007017903. Skilled person can make an expression vector comprising novel construct of the present invention by the techniques known in the art. Preferably, the present invention provides a DNA plasmid vector pVAX1 carrying either full-length S gene or S1 gene region of 2019-nCoV spike-S protein along with a gene encoding IgE signal peptide. Alternatively, t-PA signal peptide can be used to prepare a plasmid vector carrying either S gene or S1 gene. Current invention also provides method of making vectors of the present invention. Further, current invention provides injection of the DNA construct or the DNA plasmid vector into muscle cells. It can be done by the standard needle based techniques known in the art. Such transfection is preferably carried out by a needle-free injection or by an electroporator system. A needle-free injection system (NFIS) is known to the person skilled in the art. The use of NFIS eliminates use of needles during vaccine administration thus eliminates the costs and risk associated with sharp-needle waste. Further, NFIS doesn't required external energy sources such as gas cartridges or electricity and spring provides the power for the device. These injector create a stream of pressurized fluid that penetrates upto 2 mm in skin at high velocity resulting in uniform dispersion and higher uptake of DNA molecules in cells compare to needle and syringe where the intradermal accumulation is inconsistent across individuals (as measured by bleb size) and varies among animal species. One of the needle free injection system can be Pharmajet® device. The said device is currently used commercially for certain vaccinations such as vaccination of—MMR vaccine, IPV vaccine and Flu vaccine. Further the device has been evaluated in clinical trial for DNA vaccines (6). Among electroporation devices, Cliniporator®, Trigrid Delivery System or Cellectra® devices can be used. These devices have already been used widely in several DNA delivery trials ranging from gene therapy to infectious disease prevention (7, 8 and 9). The plasmid DNA construct was injected preferably intramuscularly into muscle cells. Direct administration of the DNA construct into muscles cells allows it to remain in the nucleus as an episome without getting integrated into the host cell DNA. The inserted cloned DNA in the episome may direct the synthesis of the encoded full-length S protein antigen or S1 region of S protein antigen using host cell protein translation machinery. The DNA construct prepared according to the current invention can also be administered into the subject via other parenteral routes. Such parenteral route is selected from subcutaneous, intravenous, intradermal, transcutaneous, and transdermal, as well as delivery to the interstitial space of a tissue. The DNA construct may be adapted for parenteral administration, for instance in the form of an injectable that may be sterile and pyrogen free. In one of the embodiments, the present invention provides an immunogenic composition comprising DNA construct or the vector comprising DNA construct of the present invention. Such immunogenic composition may optionally include a gene encoding IgE signal peptide or a gene encoding t-PA signal peptide. The current invention further provides method of making immunogenic composition comprising the DNA construct or the DNA construct based vector of the current invention. The said method includes (i) preparation of DNA construct or preparation of vector comprising DNA construct and (ii) addition of suitable adjuvant and/or suitable pharmaceutical excipient into the preparation of step (i). Suitable pharmaceutical excipient is selected from buffer, stabilizer, adjuvant and suitable combination thereof. The preparation of DNA construct includes construction of DNA construct encoding either S protein or S1 region of S protein of SARS-CoV-2 optionally with a gene encoding IgE signal peptide. The vector used in the construction of DNA construct encoding S protein or S1 region of S protein of SARS-CoV-2 is preferably pVAX1. The immunogenic composition prepared according to the present invention is administered parenterally into the subject. Such parenteral route is selected from intramuscular, subcutaneous, intravenous, intraperitoneal, intradermal, transcutaneous, and transdermal, as well as delivery to the interstitial space of a tissue. The immunogenic composition may be adapted for parenteral administration, for instance in the form of an injectable that may be sterile and pyrogen free. In a preferred embodiment, the present invention provides DNA vaccine comprising the DNA construct or the vector comprising the DNA construct of the present invention. The said DNA construct of the DNA vaccine comprising a gene encoding S protein or a gene encoding S1 region of S protein of SARS-CoV-2. Such vaccine may optionally include a gene encoding IgE signal peptide. The vaccine may include a SARS-CoV-2 antigenic peptide, a SARS-CoV-2 antigenic protein, a variant thereof, a fragment thereof, or a combination thereof. The vaccine prepared according to the present invention is administered parenterally into the subject. Such parenteral route is selected from intramuscular, subcutaneous, intravenous, intraperitoneal, intradermal, transcutaneous, and transdermal, as well as delivery to the interstitial space of a tissue. The vaccine may be adapted for parenteral administration, for instance in the form of an injectable that may be sterile and pyrogen free. In one of the embodiments, the vaccine or the immunogenic composition prepared according to the current invention include different formulations comprising DNA or vector prepared according to the present invention. The immunogenic composition of the present invention can be prepared in water or saline. The immunogenic composition or formulation according to the current invention are prepared with different buffers having different ionic strength, stabilizer(s), adjuvant(s) and optionally other suitable pharmaceutical excipient(s). In a preferred embodiment of the present invention, the immunogenic composition comprises of (a) buffer; (b) stabilizer(s) selected from free radical scavenger and/or metal ions chelators; (c) other pharmaceutical excipient(s) selected from bupivacaine hydrochloride and/or sugars selected from Vi-polysaccharide, zymogen and/or chitosan; and (d) adjuvant(s) selected from aluminum hydroxide gel, bacterially derived adjuvant, lipophilic adjuvant, hydrophilic adjuvant, Complete Freund's adjuvant (CFA), Incomplete Freund's adjuvant (IFA), mono phosphoryl lipid A, beta-sitosterol and suitable combination thereof. Various immunogenic compositions comprising pDNA are prepared using a buffer having different ionic strength or different pH.

Preferably, the formulation or immunogenic composition is pDNA liposomal formulation. In one of the embodiments, the formulation is prepared by lipid entrapment method using cationic lipids (CL) comprising of one or more lipids selected from DOTMA (1, 2-di-O-octadecenyl-3-trimethylammonium propane) and DOTAP (N-[1-(2, 3-Dioleoyloxy) propyl]-N, N, N-trimethylammonium methyl-sulfate). Said formulation can be used for plasmid DNA delivery. CLs are used as carriers of pDNA as they form a complex with pDNA and such a complex transports pDNA to the cytosol. The formation of pDNA liposome depends on the molar ratio of cationic lipid nitrogen (N) to pDNA phosphate (P) which is termed here as N/P ratio. The N/P ratio influences the final characteristics of the pDNA liposomes such as size, surface zeta potential and reproducibility and thereby reflecting their efficiency following transfection. pDNA liposomes are often prepared by adding helper lipid(s). Helper lipid(s) is a neutral lipid(s) which is incorporated to enhance transfection. Preferred formulation according to the current invention contains N/P ratio selected from 1, 2 and 3. Molar ratio of the cationic lipid to the helper lipid according to the current invention is 1:1 for the formulations which contain helper lipid(s).

pDNA liposome formulation can comprise of single vial formulation or two-vial formulation. Single vial formulation can be liquid injection or lyophilized powder for injection. Two-vial based formulation include one vial containing pDNA and the other vial containing lipid dispersion. Both the vials can be mixed at the time of administration.

More preferably, the formulation or immunogenic composition of the present invention is liquid formulation comprising pDNA with phosphate buffer saline. The said pDNA construct comprising gene encoding full-length S gene. The said immunogenic composition or liquid formulation comprising pDNA with phosphate buffer saline can also be referred as DNA vaccine of the present invention which is final formulated drug product. Final drug product prepared according to the present invention is at least stable for 6 months at 5±3° C. and further stable for 3 months at 25±2° C.

The vaccine prepared according to the current invention induce humoral and/or cellular immune response into the subject against coronavirus, preferably SARS-CoV-2. In one of the embodiments, the vaccine prepared according to the current invention induces generation of anti-viral CD8⁺ T cell responses. The elicited CD8⁺ T cell response can be polyfunctional. The induced cellular immune response can include eliciting a CD8⁺ T cell response, in which the CD8⁺ T cells produce IFN-γ, TNF-α, IL-2, or a combination of IFN-γ and TNF-α. The immune response can be measured by ELISA as described herein the application. Change in antibody levels after immunization in healthy subjects is detected. Subjects responding to vaccine can be marked as seroconverted. Seroconversion is defined herein as four fold rise in antibody titers against S or S1 protein from the baseline or placebo group. DNA vaccine prepared according to the current invention was evaluated in-vivo in different animal models and has demonstrated ability to elicit immunogenic response against SARS-CoV-2, S-antigen in different animal species. The serum IgG levels against spike antigen in mice were maintained even after three months post last dosing suggesting a long-term immune response generated by the DNA vaccine of the current invention. This also indicates that DNA vaccine of the present invention may induce robust secondary anamnestic immune response upon re-exposure, generated by balanced memory B and helper T cells expression. Further, cytokine response can also be measured including Th-1 and Th-2 cytokines but not limited to IFN-γ, TNF-α, IL-2, IL-4, IL-5, IL-6 and IL-10. In one of the preferred embodiments, the vaccine prepared according to the current invention provides significant increase in IFN-γ expression indicating strong Th1 response.

The vaccine of the current invention can induce generation of coronavirus neutralizing antibodies into the subject. In addition, it can induce generation of immunoglobulin G (IgG) antibodies that are reactive with the SARS-CoV-2 spike antigen. The method for testing neutralizing antibodies can be pseudovirion assay using lentivirus vector or using VSV vector or using measles vector system. Serum neutralizing antibody (Nab) titres following DNA vaccination, can be tested by micro-neutralization assay and Genscript neutralizing antibody detection kit. The Nab titre values tested by both methods demonstrated that the DNA vaccine of the current invention generates robust response and neutralizes the SARS CoV-2 virus conferring protective immunity against infection.

The present invention provides method of treating or method of preventing coronavirus, preferably SARS-CoV-2 or its related disease by administration of suitable therapeutic dose of the DNA vaccine prepared according to the current invention. The vaccine can be used to protect against any number of strains of SARS-CoV-2 thereby treating, preventing and/or protecting against SARS-CoV-2 based pathologies. The DNA vaccine can be administered in single, double or three dose regimen separated by 14-28 days between each dose.

In one of the embodiments, the DNA vaccine of the current invention is safe and tolerated when administered intradermally as well as intramuscularly. DNA vaccine of the current invention is safe up to 2 mg when administered intradermally and 6 mg dose intramuscularly in rats and rabbits. No treatment related effects where observed in any of the animal group. Further histopathology examination demonstrates no gross lesions in visceral organs.

In one of the embodiments, the DNA vaccine of the present invention is co-delivered with cytokines to enhance the production of a Th1 immune response beneficial in a viral infection. One such cytokine can be IFN-alpha or PEGylated IFN-alpha or IFN beta which can be co-delivered with the vaccine of the current invention to enhance the production of a Th1 immune response beneficial in a viral infection.

### EXAMPLES

The following examples are put forth so as to provide those of ordinary skills in the art with a disclosure and description of how DNA construct, its composition, its vaccine and the methods claimed herein are performed. They are intended to purely exemplify only and are not intended to limit the scope of the disclosure. The other DNA constructs of the present invention can be developed using method as described in provided examples with some modifications. Such modifications are known to the person skilled in the art.

### Example 1: Synthesis or Isolation of Full-Length S Gene or S1 Gene of SARS-CoV-2

The amino acid sequences of full-length S gene (SEQ ID No.: 1) and S1 region (SEQ ID No.: 2) of S gene was taken from NCBI (MN908947.2.). The genes of interest were codon optimized for expression in humans and chemically synthesized by GeneArt, Germany. Codon optimized nucleotide sequences of S gene and S1 region of S gene are highlighted in bold in the given nucleotide sequences herein above.

### Example 2: Construction of Vector Encoding S Gene or S1 Gene and IgE/t-PA Signal Peptide

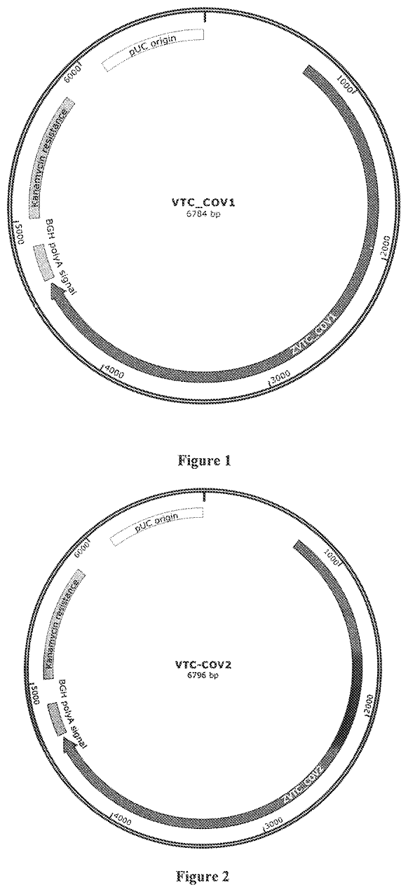

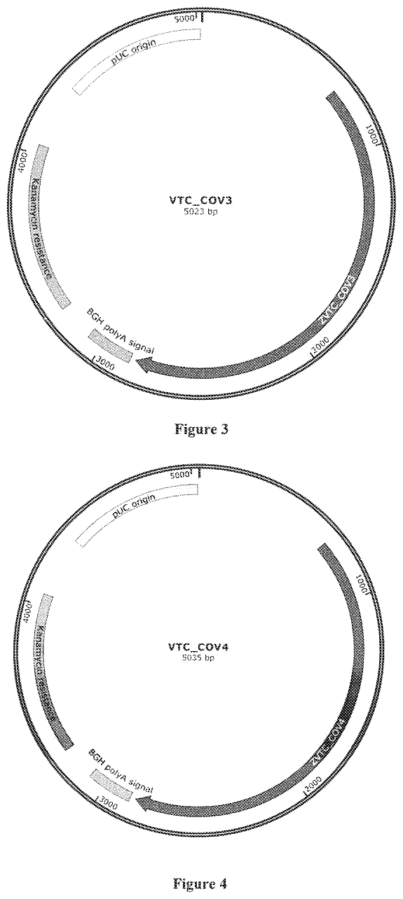

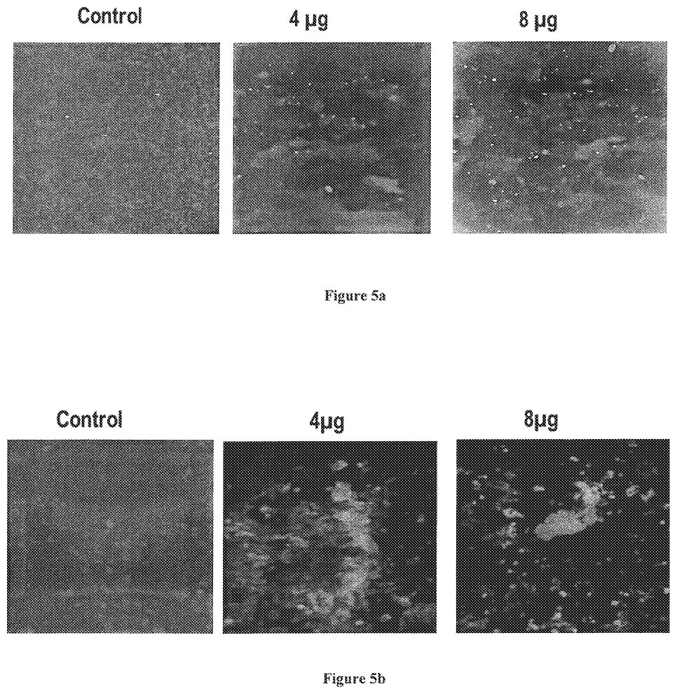

All chemically synthesized genes, full-length S gene with IgE leader sequence (SEQ ID No.: 3 and SEQ ID No.: 4), full length S gene with t-PA leader sequence (SEQ ID No.: 5 and SEQ ID No.: 6), S1 region of S gene with IgE leader sequence(SEQ ID No.: 7 and SEQ ID No.: 8), S1 region of S gene with t-PA leader sequence(SEQ ID No.: 9 and SEQ ID No.: 10) were digested with NheI and ApaI restriction sites and inserted into the pVAX1 vector digested with same set of restriction enzymes. The presence and integrity of gene was confirmed by Sanger sequencing and restriction enzyme profiling of the vector. The pVAX1 vector carrying full length S gene with IgE leader sequence was named as ZVTC_COV1 (FIG. 1). The second vector carrying full length S gene with t-PA leader sequence was named as ZVTC_COV2 (FIG. 2). Third vector carrying S1 region of S gene with IgE leader sequence was named as ZVTC_COV3 (FIG. 3) and fourth vector carrying S1 region of S gene with t-PA leader sequence was named as ZVTC_COV4 (FIG. 4). In the same manner, the plasmid DNA construct encoding S protein of SARS-CoV-2 with 2P substitution or with Hexapro substitution can be prepared by following the method as illustrated herein examples 1 and 2. The plasmid DNA constructs were transformed in DH5-α™ chemically competent cells. After heat shock transformation step, E. coli clones carrying the plasmid DNA constructs were isolated by plating on LB agar plate containing Kanamycin antibiotic. Single colonies were picked and inoculated in flasks containing LB broth from Hi-Media with Kanamycin. Flasks were incubated in 37° C. incubator shaker at 225 rpm for 20 Hrs. Culture from each clone was used for plasmid isolation using miniprep plasmid isolation kit. Restriction digestion was carried out with BamH1, Nhe1 and Apa1 for all constructs to check expected band releases of inserts to select the positive clones. Positive clones were selected for preparation of glycerol stocks and stored at −70° C.

### Example 3: In-Vitro Expression Analysis of the DNA Constructs

In-vitro expression of DNA vaccine candidate was checked by transfection of the same in Vero cell line. For transfection experiments, Vero cells were seeded at density of 3×10⁵ cells/ml in 6 well plates and kept in CO₂ incubator to attain 80-90% confluency. After 24 Hrs, once the cells reached the desired confluency, transfection was carried out in OptiMEM serum free medium with Lipofectamine 2000 reagent (Thermo Fisher). Two different concentrations (4 μg and 8 μg) of DNA construct was used for transfection experiments. After transfection, media was replenished with fresh DMEM media (Biowest) containing FBS. After 72Hrs, plates were fixed with 1:1 acetone and methanol. Anti-S1 rabbit polyclonal antibody (Novus) was added to each well and incubated for 1Hr followed by incubation with FITC labelled anti-rabbit antibody (Merck). Fluorescence images were captured using an inverted microscope (ZeissAX10). Fluorescence images showing expression of S protein and S1 protein after transfection of Vero cells with DNA construct or empty plasmid (control) by immunofluorescence are given here as FIGS. 5a and 5b, respectively.

### Example 4: Transfection of Vectors into Host Cell

This example describes the transfection of CHO host cell lines with vector encoding S gene or S1 gene. Transfections were carried out in CHO cell lines using 2 different methods.

(1) Transfection in Freestyle CHO-S Cells (Invitrogen) Using Electroporation Method

Freestyle CHO-S cells (Invitrogen) were used as a host for transfection. Cells were routinely cultured in Power CHO 2 CD media from Lonza. Cells were seeded ˜24 hours prior to transfection to have them growing in exponential phase. Neon Transfection system (Invitrogen) was utilized for performing transfection via electroporation following pre-optimized conditions. Following transfection, cells were plated in 24 well plate containing 1 ml pre-warmed media from Lonza. Cells were incubated in a humidified incubator at 37° C. in the presence of 5% CO₂. The cell number of the pool was monitored regularly over a period of 1-3 weeks. Transfected pools were further transferred to 6 well plate and then into T-flasks/culti-tubes. Transfected pool cells and supernatant were stored for expression analysis of S gene or S1 gene.

(2) Transfection in ExpiCHO S™ Cells (Gibco, ThermoFisher) Using Lipid Based Method

ExpiCHO S™ Cells were routinely maintained in ExpiCHO™ expression medium. On the day prior to transfection, split the ExpiCHO ST™ Cells to a final density of 3×10⁶ cells/ml. On the next day, transient transfections were performed using ExpiFectamine™ CHO reagent according to the manufacturer's protocol (Gibco, ThermoFisher). Post transfection, ExpiFectamine™ CHO Enhancer was added and cells were shifted to 32° C. on Day 1. Feeding was done on day 1 and day 5. The culture were harvested when cell viability reached <50%. Further, cells and supernatant were stored for expression analysis.

### Example 5: Preparation of Immunogenic Composition or Formulation Comprising pDNA

### Preparation of Immunogenic Composition—

Purified plasmid DNA encoding S gene with IgE signal peptide (represented herein as SEQ ID NO. 4) was added to sterile filtered phosphate buffered saline under constant stirring as per the final formulation concentration. After ensuring homogeneity, the formulated bulk was filter sterilized using 0.2μ filter. The blend was filled in vials and was visually inspected. Vials were segregated and samples for Quality Control were collected for testing. Remaining vials were labelled & packed in mono carton & stored at 2 to 8° C.

TABLE 2
Stability of DNA vaccine (Drug product under) under
Real time Conditions (5 ± 3° C.)

| Test | Acceptance Criteria | Initial | 1 M | 3 M | 6 M |
|---|---|---|---|---|---|
| Identity | Linearization should be | Complies | Complies | Complies | Complies |
| | observed when digested | | | | |
| | Restriction enzymes | | | | |
| DNA Purity | 1.8-2.0 | 2.0 | 2.0 | 2.0 | NS |
| DNA form for | ≥80% | 90.7% | 88.8% | 88.4% | 89.3% |
| supercoiled | | | | | |
| plasmid content | | | | | |

TABLE 3
Stability of Vaccine (Drug product under) under Accelerated
Conditions (25 ± 2° C.)

| Test | Acceptance Criteria | Initial | 1 M | 2 M | 3 M |
|---|---|---|---|---|---|
| Identity | Linearization should be | Complies | Complies | Complies | Complies |
| | observed when digested | | | | |
| | Restriction enzymes | | | | |
| DNA Purity | 1.8-2.0 | 2.0 | 2.0 | 2.0 | 2.0 |
| DNA form for | ≥80% | 90.7% | 86.9% | 85.2% | 83.3% |
| supercoiled | | | | | |
| plasmid content | | | | | |

The stability data shows that DNA vaccine prepared according to the present invention can be stored at 2-8° C. for long term and further at 25° C. for three months. In the context of a pandemic outbreak, the stability profile of a vaccine plays a vital role easy deployment and distribution for mass vaccination.

### Single Vial Formulation:

Lipid dispersion was prepared by thin film hydration or ethanol injection method. To the said lipid dispersion, pDNA was added and mixed to form pDNA liposomes. pDNA liposomes were de-aggregated by means of bath sonication or homogenization or a suitable method. pDNA liposomes were then administered by IM injection. Further, the pDNA liposome formulation can be lyophilized to stabilize the prepared formulation. pDNA liposome formulation can be lyophilized by adding cryoprotectant such as sucrose, lactose or mannitol. Afterwards, the product was subjected to lyophilization. At the time of administration, pDNA liposome formulation vial can be reconstituted with sterile water for injection or a suitable buffer. The reconstituted pDNA liposomes are then administered by IM injection.

### Two Vial Formulation:

Preparation of Vial 1—Lipid dispersion was prepared by thin film hydration or ethanol injection method. Size of the lipid dispersion was reduced to below 200 nm. The lipid dispersion was then filtered through 0.2μ sterile grade filter and filled in Vial 1.

Preparation of Vial 2—It contains sterile filtered pDNA solution.

pDNA liposome are prepared by mixing content of Vial 1 and Vial 2 at controlled room temperature. pDNA liposomes are then administered by IM injection.

### Example 6: Animal Immunization with DNA Vaccine

The immunogenicity study for the DNA vaccine was carried out in inbred BALB/C mouse, guinea pig, and New Zealand white rabbit model after having ethical approval from Institutional Animal Ethics Committee. BALB/c mouse (five to seven-week-old), guinea pigs (five to seven-week-old) and New Zealand White rabbits (six to twelve-week-old) were used in this study. For mouse intradermal immunization, on day 0; 25 and 100 μg of DNA vaccine was administered to the skin by using 31 gauge needle. Animals injected with empty plasmid served as vehicle control. Two weeks after immunization, animals were given first booster dose. Similarly all mice were given second booster dose two weeks after first booster dose. For guinea pig study, intradermal immunization was carried out using same dosing and schedule. In rabbits, DNA vaccine was administered to the skin by using needle free injection system (NFIS) at 500 μg dose at same 3 dose regimen and schedule. Blood was collected from animals on day 0 (before immunization) & 28 (after 2 dose) and on day 42 (after 3 dose) for immunological assessments from sera samples. In mouse model long term immunogenicity of the vaccine was assessed for up to day 126. Further, IFN-γ response from splenocytes at day 0, 28, and 42 were assessed.

### Example 7: Immunogenicity Study in Animal Model with Different Immunogenic Compositions

The DNA vaccine prepared according to the current invention was tested at its different dose strength and with different immunogenic composition. 25 to 100 μg of SARS-CoV-2 DNA vaccine comprising plasmid DNA construct comprising gene encoding full-length S gene was delivered to mice or guinea pigs by either IM/ID/SC route. 25 μg and 100 μg of SARS-CoV-2 DNA vaccine was administered intradermally to Balb/c mice and Guinea pigs as described in following table 4.

TABLE 4
Study plan for immunogenicity study of DNA vaccine

| | | | | Dosing | Bleeding time |
|---|---|---|---|---|---|
| Species | Age | Dose | Route | regimen | points |
| Balb/c | 5-7 | 25 & | Intradermal | 0 Day, 14 day, | 14 Day, 28 Day, |
| | Weeks | 100 μg dose | | 28 Day | 35 Day, 42 Day |
| Guinea | 5-8 | 25 & | Intradermal | 0 Day, 14 day, | 14 Day, 28 Day, |
| Pig | weeks | 100 μg dose | | 28 Day | 35 Day, 42 Day |

0.1 mL/0.05 ml/0.5 ml of vaccine formulation was injected through IM/ID/SC route in mice and guinea pigs on day 0. Same procedure of immunization was repeated on day 14 and day 28. Animals were observed for up to 56 days. To assess the immunogenicity by ELISA and other methods, animals were bled on day 0, 14, 28 (before immunization) and also on day 42 and day 56. Sera samples were tested against recombinant S1 antigen of SARS-CoV-2 using standard ELISA as described in example 8.

### Example 8: Analysis of Antibody Response to S or S1 Protein by ELISA

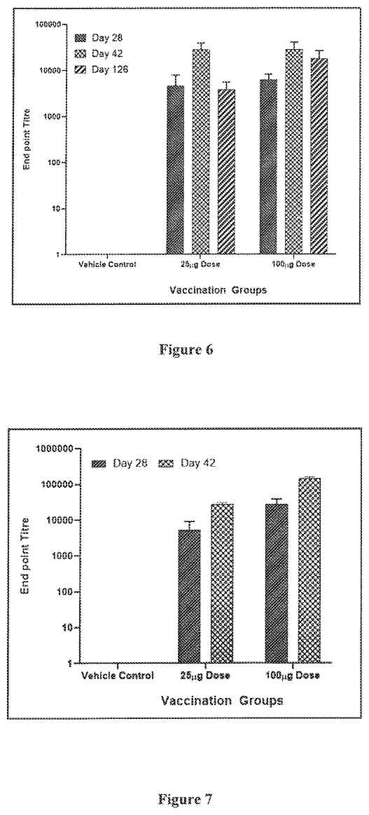

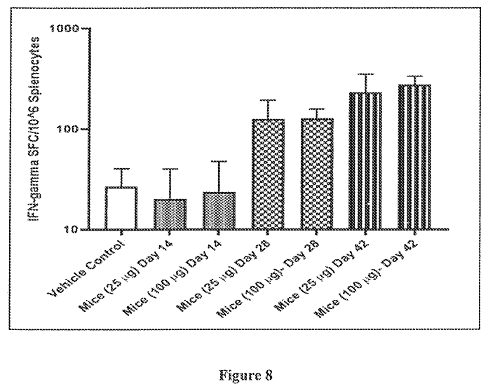

Indirect ELISA was done to detect IgG antibodies against S or S1 protein following vaccination. Four fold rise in IgG antibody titers was considered as seroconversion. 96 well plates were coated with 50 ng/well of recombinant purified S1 spike protein of SARS-CoV-2 (Acro, USA) in phosphate-buffered saline (PBS) overnight at 4° C. Plates were washed three times then blocked with 5% skimmed milk (BD Difco) in PBS for 1 Hr at 37° C. Bovine serum albumin (BSA) in PBS can also be used in place of skimmed milk in PBS. Plates were then washed thrice with PBS and incubated with serial dilutions of mouse and guinea pig sera and incubated for 2 hours at 37° C. Plates were again washed thrice and then incubated with 1:2,000 dilution of dilution of horse radish peroxidase (HRP) conjugated anti-mouse IgG secondary antibody (Sigma-Aldrich) or 1:5,000 dilution of horse radish peroxidase (HRP) conjugated anti-guinea pig IgG secondary antibody (Sigma-Aldrich) and incubated for 1 hour at 37° C. Incubation can be performed at RT in place of 37° C. After final wash plates were developed using TMB Peroxidase Substrate (KPL) and the reaction stopped with TMB Stop Solution (1 N H₂SO₄). Plates were read at 450 nm wavelength within 30 minutes using a multimode reader (Molecular Devices, USA). Immunization with DNA vaccine candidate by intradermal route elicited significant serum IgG responses against the S protein in doses-dependent manner in BALB/c mice and guinea pigs with mean end point titres reaching ˜28000 in BALB/C mice and ˜140000 in guinea pigs respectively on day 42 after 3 doses (FIGS. 6 and 7). Long term antibody response was studied in mice almost 3.5 months after the last dose and a mean end point IgG titres of ˜18000 was detected (FIG. 6) suggesting sustainable immune response was generated by DNA vaccine candidate.

### Example 9: Analysis of Cellular Immune Response by Measuring Cytokine Production

Two weeks following the final injection, single-cell suspension was prepared from spleens or monocytes. 2-3×10⁵ cells per well in RPMI-1640 medium (Sigma) supplemented with 10% FBS were seeded in a 96-well plate in triplicate. Cultures were stimulated under various conditions for 60 h at 37° C. and 5% CO₂: 5 μg/ml Concanavalin A (positive control), 5 μg/ml purified S antigen (specific antigen), 5 μg/ml BSA (irrelevant antigen) or medium alone (negative control). 20 μl of CellTiter 96 Aqueous One Solution Reagent (Promega, USA) was added into each well according to the manufacturer's protocol. Following 4 h incubation at 37° C., absorbance was read at 490 nm. Proliferative activity was estimated using the stimulation indexes (SI). SI is defined as the mean OD 490 of antigen-containing wells divided by mean OD 490 of wells without antigen. Cytokine response was measured in serum samples as well as in supernatant obtained after cell proliferation. Cellular immune responses to SARS-CoV-2 was assessed by ELISA/FACS/ELiSpot assay. Assays were performed according to the standard operating procedure provided with cytokine detection kits.

### IFN-γ ELISPOT Assay

For IFN-γ ELISPOT assay, spleens from immunized mice were collected in sterile tubes containing RPMI 1640 (Thermoscientific) media supplemented with 2× Antibiotic (Antibiotic Antimycotic, Thermoscientific). Cell suspensions were prepared by crushing the spleen with disk bottom of the plunger of 10 ml syringe (BD) in sterile petri plates. Then 5-10 ml of RPMI-1640 medium supplemented with 1× Antibiotic was added to it and the contents were mixed for homogeneity. Dishes were kept undisturbed for 2 min and the clear supernatant was pipetted out slowly into cell strainer (BD). The filtrate was collected in sterile tubes and the cells were pelleted by centrifugation at 4° C. for 10 min at 250×g in a centrifuge (Thermo Scientific). The pellet containing red blood cells (RBCs) and splenocytes were collected. 2-3 ml RBC Lysing Buffer (Invitrogen) was added to the pellet containing splenocytes and incubated at room temperature for 5-7 min. After incubation RPMI 1640 supplemented with 10% FBS (Biowest) and 1× antibiotic was added thrice the volume of RBC Lysing Buffer added previously. The pellets were washed with RPMI 1640 supplemented with 10% FBS and 1× antibiotic twice and were re-suspended in RPMI 1640 medium containing 10% FBS and 1× antibiotic and adjusted to a density of 2.0×10⁶ cells/ml. The % well Mouse IFN-γ ELISPOT kit (CTL, USA) plates pre-coated with purified anti-mouse IFN-γ capture antibody were taken out blocked with RPMI+10% FBS+1× antibiotic for 1 Hr in CO₂ incubator. The plate were then washed with PBS once and then 200,000 splenocytes were added to each well and stimulated for 24 Hrs at 37° C. in 5% CO₂ with pool of 12-mer peptides (GenScript) at a concentration of 5.0 μg/well spanning the entire SARS-CoV-2 S protein along with Negative control (RPMI 1640 supplemented with 10% FBS and 1× antibiotic and positive control (Concanavalin A. 1 μg/well). After stimulation, the plates were washed with PBS followed PBS containing 0.05% tween and spots were developed as per the manufacturer's instructions provided along the kit. The plates were dried and the spots were counted on ELISPOT Reader S6 Versa, (CTL USA) and analysed with Immunospot software version 7.0.

### Cellular Immune Response to DNA Vaccine Candidate

T cell response against SARS-CoV-2 spike antigen was studied by IFN-γ ELISpot assay. Groups of BALB/c mice were sacrificed at day 14, 28, 42 post-DNA vaccine administration (25 and 100 μg dose). Splenocytes were harvested, and a single-cell suspension was stimulated for 24 h with pools of 12-mer overlapping peptides spanning the SARS-CoV-2 spike protein. Significant increase in IFN-γ expression, indicative of a strong Th1 response, of 200-300 SFC per 10⁶ splenocytes against SARS-CoV-2 spike peptide pool was observed for both the 25 and 100 μg dose in post 42 day immunized mice splenocytes (FIG. 8).

### Example 10: Analysis of Virus Neutralization by Virus Neutralization Assays Using Wildtype SARS-CoV-2

Micro-neutralization test (MNT) was performed. The virus was obtained from the BEI resources, USA (Isolate USA-WA 1/2020), passaged and titrated in Vero-E6 cells. The sera samples collected from immunized animals were heat-inactivated at 56° C. for 30 min followed by two fold serial dilution with cell culture medium. The diluted sera samples were mixed with a virus suspension of 100 TCID₅₀ in 96-well plates at a ratio of 1:1 followed by 1 Hr incubation. This is followed by 1 Hr adsorption on Vero-E6 cells seeded 24 Hrs prior to experiment in 96 well tissue culture plate (1×10⁴ cells/well in 150 μl of DMEM+10% FBS). The cells were subsequently washed with 150 μl of serum free media and 150 μl of DMEM media supplemented with 2% FBS, followed by incubation for 3-5 days at 37° C. in a 5% CO₂ incubator. Cytopathic effect (CPE) was recorded under microscopes in each well. Neutralization was defined as absence of CPE compared to virus controls. Neutralizing antibodies were elicited by DNA vaccine candidate in mice, guinea pigs and rabbits. Sera from DNA vaccine candidate immunized BALB/c mice could neutralize wild SARS-CoV-2 virus strains with average MNT titres of 40 and 160 at day 42 with 25 and 100 μg dose regimens respectively (Table 5).

### Example 11: Detection of Neutralizing Antibodies by Competitive Inhibition ELISA

Competitive inhibition ELISA was performed using SARS-CoV-2 neutralization antibody detection kit (Genscript). The kit detects circulating neutralizing antibodies against SARS-CoV-2 that block the interaction between the receptor binding domains of the viral spike glycoprotein (RBD) with the ACE2 cell surface receptor.

Different animal sera samples serially diluted with dilution buffer provided in the kit. The diluted sera samples were incubated with HRP conjugated RBD at 1:1 ratio for 30 min at 37° C. along with positive and negative controls. The sera and HRP conjugated RBD mix was then added to the ELISA plate pre-coated with the ACE2 protein. After that, plates were incubated for 15 min at 37° C. followed by washing four times with wash solution provided in the kit. After washing steps, TMB solution was added to the well and incubated in dark for 15 min at room temperature, followed by addition of stop solution. Plates were read at 450 nm. Inhibition concentration (IC50) of sera sample was calculated by plotting the percentage competition value obtained for each dilution verses serum dilution in a non-linear regression curve fit using Graph pad Prism 8.0.1 software. Using Genscript neutralizing antibody detection kit average IC50 titres of 82 and 168 were obtained at day 42 with 25 and 100 μg dose regimens respectively. Further, neutralizing antibodies were also detected in long term immunogenicity studies in BALB/c mice. Significant rise in neutralizing antibodies levels were also observed in guinea pigs and rabbits (Table 5).

TABLE 5
Sera neutralizing antibody titres after DNA vaccine administration
to BALB/c mice, Guinea pigs and New Zealand White Rabbits

| | Immunization | | Neutralization | Neutralization |
|---|---|---|---|---|
| Species | regimen | Neutralization Assay | Titre Day 28 | Titre Day 42 |
| BALB/c | 25 μg | Micro-neutralization | 20 | 40 |
| mice | Days 0, 14, 28 | (SARS-CoV-2 USA- | | |
| | | WA1/2020)-MNT₁₀₀ | | |
| | 100 μg | Micro-neutralization | 40 | 160 |
| | Days 0, 14, 28 | (SARS-CoV-2 USA- | | |
| | | WA1/2020)-MNT₁₀₀ | | |
| | 25 μg | Genscript ® Neutralization | 14 | 82 |
| | Days 0, 14, 28 | Assay-IC₅₀ | | |
| | 100 μg | Genscript ® Neutralization | 71 | 168 |
| | Days 0, 14, 28 | Assay-IC₅₀ | | |
| Guinea Pigs | 25 μg | Micro-neutralization | 20 | 80 |
| | Days 0, 14, 28 | (SARS-CoV-2 USA- | | |
| | | WA1/2020)-MNT100 | | |
| | 100 μg | Micro-neutralization | 40 | 320 |
| | Days 0, 14, 28 | (SARS-CoV-2 USA- | | |
| | | WA1/2020)-MNT100 | | |
| | 25 μg | Genscript ® Neutralization | 14 | 129 |
| | Days 0, 14, 28 | Assay-IC50 | | |
| | 100 μg | Genscript ® Neutralization | 21 | 371 |
| | Days 0, 14, 28 | Assay-IC50 | | |
| New Zealand | 500 μg injected | Genscript ® Neutralization | 30 | 108 |
| White | by NFIS | Assay-IC50 | | |
| Rabbits | Days 0, 14, 28 | | | |

### Example 12: Analysis of Neutralizing Antibodies by Pseudovirion Assay

The pseudoviruses are be produced and titrated. For this pseudovirus system, the backbone is provided by VSV pseudotyped virus/lenti pseudotype virus or measles pseudotype virus that packages expression cassettes along SARS-CoV-2 spike (S) protein.

293T cells are transfected with plasmid vector carrying the SARS-CoV-2 spike protein and helper plasmid using lipofectamine following the manufacturer's instruction. Two hours after infection, cells are washed with PBS three times, and then new complete culture medium will be added. 2-15 days (depending upon the pseudovirus type used) of the post infection, SARS-CoV-2 pseudoviruses containing culture supernatants are harvested, filtered (0.45-μm pore size) and stored at −70° C. in 2-ml aliquots until use. The 50% tissue culture infectious dose (TCID₅₀) of SARS-CoV-2 pseudovirus are determined using a single-use aliquot from the pseudovirus bank. All stocks are used only once to avoid inconsistencies that could have resulted from repeated freezing-thawing cycles. For titration of the SARS-CoV-2 pseudovirus, a 2-fold initial dilution is made in 96-well culture plates followed by serial 3-fold dilutions (nine dilutions in total). The last column is served as the cell control without the addition of pseudovirus. Then, the 96-well plates are seeded with trypsin-treated mammalian cells adjusted to a pre-defined concentration. After 2-9 days (depending upon the pseudovirus type used) of the incubation in a 5% CO₂ environment at 37° C., the positive wells are determined. The 50% tissue culture infectious dose (TCID₅₀) is calculated using the Reed-Muench method.

### Example 13: Manufacturing Process of DNA Plasmid

a) Cell Revival and Fermentation:

Cell culture from working cell bank was used to inoculate pre-seed media. Pre-seed media was incubated at 30±1 C to achieve an OD of ≥1.5. Pre-seed media was inoculated in to seed fermentation media and incubated at 30±2° C. in seed fermenter. After the target optical density was reached at about ≥2.0, seed fermentation media was used to inoculate the production fermenter. The culture is incubated at 30-42° C., pH of 7.0±0.3 with dissolved oxygen concentration maintained at 0-100% by a cascade of agitation and oxygen enrichment. The fermentation was terminated after bacterial growth was reached to the stationary phase. Harvested broth was centrifuged and cell pellet was stored at or below −70° C. still lysis was performed. Lysis of the cells was done by chemical method with solution containing 0.2M NaOH and 1% SDS. The pH of the lysed culture broth was adjusted to approximately pH 5-13. Post lysis, concentration, cell lysate was clarified by continuous flow centrifugation or depth filtration and clarified lysate was collected as filtrate.

b) Purification:

Purification process initiated with cell lysis by suspension in resuspension solution with a cell to buffer ratio maintained in the range of 1:5 to 1:12. After resuspension, solution comprising 1% SDS and 0.2N NaOH was added at the same cell to buffer ratio for the cell lysis. Post lysis, chilled potassium acetate buffer was added to neutralize the pH of the solution at about pH 5.5. After neutralization, CaCl₂) was added to the same reaction mixture with a target concentration of not less than 0.5-1.0M CaCl₂ to remove RNA impurities. Post CaCl₂ treatment, reaction mixture was subjected to the clarification followed by buffer exchange by UF/DF, for reconditioning of the solution. The reconditioned solution was further subjected to anion exchange column chromatography for the removal residual RNA and other product- and process-related impurities.

The purification of clarified lysate consisted of several concentration/diafiltration operations and anion exchange chromatography. The clarified lysate was diafiltered using 100-500 kDa MWCO (kilo Dalton molecular weight cutoff) filter. Diafiltration was accomplished using AEX equilibration buffer at neutral pH. The filtrate containing DNA plasmid was purified using a weak anion resin with elution buffer in step elution mode. The operating flow rate for these runs was approximately 120-250 cm/hr. The plasmid DNA eluted from the anion exchange column was analyzed by gel electrophoresis.

Further, the anion exchange elution was diafiltered using 100-500 kDa cut-off cassettes in TFF. The temperature was maintained at 2-25° C. The filtrate contained the target DNA plasmid. The diafiltered solution was filtered through a 0.2 μm membrane filter to get purified DNA plasmid.

References incorporated in current patent application:
1. Tai et al., Characterization of the receptor-binding domain (RBD) of 2019 novel coronavirus: implication for development of RBD protein as a viral attachment inhibitor and vaccine. Cellular and Molecular Immunology (2020). http://doi.org/10.1038/s41423-020-0400-4
2. https://www.worldometers.info/coronavirus/
3. Wrapp et al., Cryo-EM structure of the 2019-nCoV spike in the prefusion conformation, Science 367, 1260-1263 (2020).
4. Tao et al., 2009: Tao P. Luo M, Pan R, Ling D, Zhou S. Tien P, Pan Z. Enhanced protective immunity against H5N1 influenza virus challenge by vaccination with DNA expressing a chimeric hemagglutinin in combination with an MHC class I-restricted epitope of nucleoprotein in mice. Antiviral research. 2009; 81(3); 253-260
5. Feng K, Zheng X, Wang R, et al. Long-Term Protection Elicited by a DNA Vaccine Candidate Expressing the prM-E Antigen of Dengue Virus Serotype 3 in Mice. Front Cell Infect Microbiol. 2020; 10:87.
6. Gaudinski M R et al., Safety, tolerability, and immunogenicity of two Zika virus DNA vaccine candidates in healthy adults: randomised, open-label, phase 1 clinical trials. Lancet. 2018; 391(10120):552-562.
7. Quaglino E et al., Chimeric DNA Vaccines against ErbB2+ Carcinomas: From Mice to Humans. Cancers (Basel). 2011 Aug. 10; 3(3):3225-41.
8. Spearman P et al., A phase 1, randomized, controlled dose-escalation study of EP-1300 polyepitope DNA vaccine against Plasmodium falciparum malaria administered via electroporation. Vaccine. 2016 Nov. 4; 34(46):5571-5578.
9. Modjarrad K et al., Safety and immunogenicity of an anti-Middle East respiratory syndrome coronavirus DNA vaccine: a phase 1, open-label, single-arm, dose-escalation trial. Lancet Infect Dis. 2019 September; 19(9):1013-1022.
10. Polack, F. P. et al. Safety and Efficacy of the BNT162b2 mRNA Covid-19 Vaccine. N Engl J Med. 383(27), 2603-2615 (2020).
11. Voysey, M. et al. Safety and efficacy of the ChAdOx1 nCoV-19 vaccine (AZD1222) against SARS-CoV-2: an interim analysis of four randomised controlled trials in Brazil, South Africa, and the UK. Lancet 397(10269), 99-111 (2021)
12. Liu, M A. “DNA vaccines: a review.” Journal of internal medicine vol. 253(4), 402-410 (2003).
13. Klinman, D. M. et al. “Contribution of CpG motifs to the immunogenicity of DNA vaccines.” Journal of immunology (Baltimore, Md.: 1950) vol. 158(8), 3635-3639 (1997).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A plasmid DNA construct comprising a gene encoding S protein of SARS-CoV-2 or a truncated gene of S protein of SARS-CoV-2 with a gene encoding signal peptide.

2. The truncated gene of S protein of SARS-CoV-2 as claimed in claim 1 is S1 region or a receptor binding domain RBD that binds to the human angiotensin converting enzyme-2(ACE-2) receptor.

3. A vector comprising a gene encoding S protein of SARS-CoV-2 or S1 region of S protein of SARS-CoV-2 with a gene encoding signal peptide.

4. The vector as claimed in claim 3 further comprising regulatory element(s) required for the expression of S gene of SARS-CoV-2 or S1 region of S gene of SARS-CoV-2.

5. The vector as claimed in claim 3 further comprising human cytomegalovirus immediate-early (CMV) promoter, bovine growth hormone (BGH) polyadenylation signal, kanamycin resistance gene or suitable combination thereof.

6. The vector as claimed in claim 3 is selected from pVAX1, pCDNA 3.1, pCDNA 4.0, pCMV and PCAGG.

7. The signal peptide as claimed in claim 1 is IgE signal peptide or t-PA signal peptide.

8. An immunogenic composition comprising DNA construct or vector as claimed in claim 1.

9. A method of making immunogenic composition as claimed in claim 8 comprising steps of:
(i) preparation of DNA construct or preparation of vector comprising DNA construct and
(ii) addition of at least one of a suitable adjuvant and a suitable pharmaceutical excipient into the preparation of step (i).

10. The suitable excipient as claimed in claim 9 is selected from buffer(s), stabilizer(s), and suitable combination thereof.

11. The DNA construct or vector as claimed in claim 1 is injected into a subject intramuscularly or intradermally.

12. The DNA construct or vector as claimed in claim 1 is injected by a needle-free injection or by an electroporator system.

13. A vaccine comprising DNA construct or vector as claimed in claim 1.

14. The vaccine as claimed in claim 13 induces humoral and/or cellular immune response into the subject.

15. The vaccine as claimed in claim 14 is co-delivered with cytokines to enhance the production of a Th1 immune response beneficial in a viral infection.

16. The gene encoding S protein of SARS-CoV-2 as claimed in claim 1, expresses S protein of SARS-CoV-2 wherein S protein of SARS-CoV-2 has proline substitution selected from K986P, V987P, F817P, A892P, A899P, A942P and suitable combinations thereof.

17. The combination as claimed in claim 16 is selected from two proline substitution (K986P, V987P) and six proline substitutions (K986P, V987P, F817P, A892P, A899P, A942P).

18. The gene encoding S protein of SARS-CoV-2 as claimed in claim 1 has nucleotide sequence from nucleotide residues from 55 to 3873 of SEQ ID NO.: 4 or its fragment or its variant thereof, nucleotide sequence from nucleotide residues from 67 to 3885 of SEQ ID NO.: 6 or its fragment or its variant thereof and nucleotide sequence from nucleotide residues from 55 to 3873 of SEQ ID NO.: 12 or its fragment or its variant thereof.

19. The gene encoding S protein of SARS-CoV-2 with leader sequence as claimed in claim 1 is selected from SEQ ID NO.: 4, a nucleotide sequence having at least 95% identity over an entire length of the nucleotide sequence as set forth in SEQ ID NO.: 4, SEQ ID NO.: 6, a nucleotide sequence having at least 95% identity over an entire length of the nucleotide sequence as set forth in SEQ ID NO.: 6, SEQ ID NO.: 12 and a nucleotide sequence having at least 95% identity over an entire length of the nucleotide sequence as set forth in SEQ ID NO.: 12.

20. The gene encoding S1 region of S protein of SARS-CoV-2 as claimed in claim 1 has nucleotide sequence from nucleotide residues from 55 to 2112 of SEQ ID NO.: 8 or its fragment or its variant thereof and nucleotide sequence from nucleotide residues from 67 to 2124 of SEQ ID NO.: 10 or its fragment or its variant thereof.

21. The gene encoding S1 region of S protein of SARS-CoV-2 with leader sequence as claimed in claim 1 is selected from SEQ ID NO.: 8, a nucleotide sequence having at least 95% identity over an entire length of the nucleotide sequence as set forth in SEQ ID NO.: 8, SEQ ID NO.: 10 and a nucleotide sequence having at least 95% identity over an entire length of the nucleotide sequence as set forth in SEQ ID NO.: 10.

22. The DNA construct as claimed in claim 1 is produced by scalable production process using batch or fed-batch method with suitable media compositions comprising of yeast extract, tryptone, glycerol and other suitable ingredients available for high density E. coli culture. Also, temperature range from 30° C. to 42° C. can be used according to the present invention to increase plasmid yield from bacterial biomass.

23. The DNA construct as claimed in claim 1 is purified by the purification process comprising one or more of the following steps: (a) lysis of host cell containing plasmid DNA; (b) clarifying the lysate by filtration to obtain clarified lysate; (c) treating lysate to remove endotoxin and other impurities; (d) purifying the treated solution of step (c) with plasmid DNA using one or more of the chromatography techniques selected from affinity chromatography (AC), ion exchange chromatography (IEC) and/or hydrophobic interaction chromatography (HIC); (e) concentrating the purified plasmid comprising of one or more following steps of (i) precipitation, (ii) diafiltration and/or (iii) lyophilization.
